(19) United States
(12) Patent Application Publication
(10) Pub. No.: US 2021/0315175 A1
(43) Pub. Date: Oct. 14, 2021

(54) METHODS OF MAKING FEMINIZED CANNABIS SATIVA SEED AND COMPOSITIONS THEREOF

(71) Applicant: FLORAGEN, LLC, Davis, CA (US)

(72) Inventors: Daniel OVADYA, Davis, CA (US); Beth SAVIDGE, Davis, CA (US)

(21) Appl. No.: 17/274,369

(22) PCT Filed: Oct. 21, 2020
(86) PCT No.: PCT/US20/56694

§ 371 Date: Mar. 8, 2021

Related U.S. Application Data
(60) Provisional application No. 62/924,456, filed on Oct. 22, 2019.

(51) Int. Cl. A01H 1/02; A01H 5/02; A01H 6/28; C12Q 1/6895

(52) U.S. Cl. CPC A01H 1/026; A01H 5/02; C12Q 2600/158; C12Q 1/6895; C12Q 2600/13; A01H 6/28

(57) ABSTRACT

The present disclosure relates to methods of producing viable Cannabis sativa seeds by inducing male flowers on a genetically female Cannabis sativa plant. The pollen produced by the induced flowers of such plant can be used to pollinate female flowers from the same plant or another female plant to produce seed. In certain embodiments, feminised Cannabis sativa seed is produced on a production scale.

### BACKGROUND

Plant breeding is the process of manipulating and selecting plant species in order to create improved genotypes and phenotypes for specific purposes in agriculture. This process often involves controlled pollinations to produce elite genetic combinations which are then selected for their unique traits or ability to solve key production challenges. Reproductive biology differs across cultivated crops and breeders require specific technologies in each crop to enable advanced breeding methods that are used for genetic improvement. Cannabis is emerging as a valuable new crop for medicinal, recreational, and industrial purposes and there is a need to improve breeders' ability to develop improved strains and seed production systems.

Cannabis sativa is one of the oldest cultivated plants, as it has many uses. The stem is a source of the strong, hemp fiber which is used to make rope, cloth, and other textiles. Oil extracts from the seed are used in food products, paint, varnishes, and lubricants. Female plants produce flowers with dense trichomes that accumulate oil that contains terpenes and cannabinoids with medicinal and euphoria-inducing properties.

Cannabis sativa is described as a wind-pollinated dioecious plant (an individual plant has only male or female flowers and only the flowers from female plants have value as a product themselves or extracts thereof) with gender being determined by heteromorphic chromosomes (X and Y): the male plant is heterogametic (XY) and produces male reproductive structures and the female is homogametic (XX) and produces female reproductive structures. This reproductive system makes breeding challenging for two main reasons.

First, crossing a genetically male plant to a genetically female plant produces seed in which approximately 50% of the progeny is male and approximately 50% is female, due to Mendelian segregation of the sex chromosomes. Since the most valuable cannabinoid compounds come from the unfertilized flower on female plants, male progeny must be identified and rogued out of any cultivation system before any pollen is formed and released. This requirement creates a range of logistical challenges that makes large-scale production expensive and inefficient. Establishment of male-free cultivation areas is the main reason that clonal propagation of female plants is the primary form of plant production today, even after decades of cultivation. Clonal selection and propagation has led to short-term gains in Cannabis phenotypes but for large-scale production, cloned plants have a range of production problems from cost, to pathogens, loss of genetics, and inability to scale. Thus, advanced seed-based breeding and production methods appear essential to the Cannabis industry's forecasted growth and long-term sustainability.

Second, because Cannabis is dioecious, it is biologically impossible to make inbred lines through sequential self-pollination, as has been commonly done in most row and vegetable crops. Advanced breeding methods often require development of near-isogenic inbred lines. Inbred lines are made to be homozygous through multiple generations of self-pollination (inbreeding) which allows for the selection of desired traits to be genetically fixed. Development of inbred lines simplifies the identification of markers and associations between phenotypes and genotypes, and allows for the precision breeding for traits, such as maturity adaptation, disease resistance, composition, and yield. Development of inbred lines also allows for the eventual selection and production of hybrid genetics which have a range of benefits for farmers and agricultural production.

Though Cannabis sativa is genetically dioecious, the species has shown some flexibility in gender phenotype and there are described examples of individual plants with both male and female inflorescences. Spinach is another dioecious crop showing hermaphrodite floral phenotypes and many species of cucurbits show a broad range of gender expression in flower morphology. Modification of sex phenotype may be induced by environmental triggers such as stress, light intensity, photoperiod, temperature or a combination of several environmental signals making it difficult to reproduce the effect. Sex expression can also be influenced by plant growth hormones, including auxins, ethylene, gibberellic acid, and cytokinins. The mode of action of phenotypic sex expression is thought to be caused by endogenous plant hormones. Thus, the modification of sex expression in plants is caused by a complex and difficult to predict interaction of environmental signals, hormones, and genetics. This complexity has made discovering and developing treatments to produce reliable changes in gender needed for large-scale seed production historically very challenging.

Ethylene inhibitors are thought to influence sex expression in a variety of plant genus. For example, ethylene inhibitors are used to induce male flowers in cucumbers, but in some watermelon types ethylene inhibitor has the opposite effect and hastens the appearance of female or pistillate flowers (Rudich and Zamski, 1985; Sugiyama et al., 1998). Ethylene inhibitors, such as silver thiosulfate (STS) (Lubell 2018; Ram 1982) and AVG (Ram and Sett, 1981) have been used to induce male flowers in Cannabis sativa, but not through a method that can be scaled for large-scale production. Additionally, there is no reported evidence of the production of viable seeds.

In general, seeds produced by using pollen from a female plant induced to produce male flowers onto female flowers of the same plant or female flowers from another female plant will result in exclusively female plants. Such seed are termed “feminized”. While Cannabis sativa seeds advertised as feminized seeds can be purchased in small quantities ($20-50/seed for indoor cultivation strains), the methods of producing such seeds have not been disclosed. Further, purchasers of such seed frequently report contamination of purported female seeds with male seeds. The controlled environment Cannabis sativa industry relies exclusively on vegetative clonal propagation for Cannabis sativa flower production from female plants. It is most desirable to grow Cannabis sativa from seed, rather than from clones to prevent the spread of disease from donor mother plants to clonal offspring. Clones have highly variable vigor and growth, with significant mortality. Based on the reports of variability of success of STS induction of male flowers, the lack of evidence for feminized seed production methods in the literature, the high price and limited availability of purportedly feminized seed to purchase, and the fact that the controlled environment Cannabis industry is still reliant on clonal propagation, there appears to be currently no production-scale method of inducing male flowers on female plants that successfully produces feminized seed.

Thus, there remains a need for methods, including high-throughput methods, of producing large numbers of feminized Cannabis sativa seeds free of contaminating male seeds.

### SUMMARY

Certain aspects of this disclosure are directed to a method of producing a genetically female Cannabis sativa seed comprising the steps of: a) inducing one or more male flowers on a genetically female Cannabis sativa plant by applying a chemical ethylene inhibitor to the plant, wherein the female plant forms one or more pollen-producing fertile male flowers; and b) producing the seed by pollinating a female flower with pollen from the induced one or more male flowers, wherein the seed produced is genetically female.

Certain aspects are drawn to a method of inducing male flowers on a genetically female Cannabis sativa plant comprising applying a chemical ethylene inhibitor to a genetically female Cannabis sativa plant, wherein the female plant forms one or more pollen-producing fertile male flowers.

Certain aspects provide for a method of producing an inbred variety of Cannabis sativa, the method comprising: (i) producing a genetically female Cannabis sativa plant with induced-male flowers, wherein the female plant comprises one or more pollen-producing fertile male flowers, by a method disclosed herein; (ii) producing seed by: (a)self-pollination of pollen from an induced-male flower onto a female flower of the same plant, or (b) cross-pollination of pollen from induced-male flowers onto female flowers of a separate but genetically identical female plant; and (iii) growing a genetically female Cannabis sativa plant from a seed produced in (ii); and optionally, (iv) repeating steps (i), (ii), and (iii) for at least two, three, four, five, six, or seven generations. Certain embodiments provide for an inbred Cannabis sativa seed, an inbred variety of Cannabis sativa, or a plant of the variety, and/or a population of inbred Cannabis sativa plants produced according to or derived from the above method.

Certain aspects provide for a method of producing reproducible hybrid Cannabis sativa seed comprising crossing two distinct inbred Cannabis sativa varieties, wherein at least one inbred variety has been produced by the above method. Certain embodiments provide for a hybrid Cannabis sativa seed, a hybrid Cannabis sativa plant, and/or a population of hybrid Cannabis sativa plants produced according to or derived from the above method.

Certain aspects provide for a method of producing a population of Cannabis sativa plants comprising: i) producing a genetically female Cannabis sativa plant with induced-male flowers, wherein the female plant comprises one or more pollen-producing fertile male flowers, produced by a method disclosed herein; (ii) producing seed by pollinating a female flower with pollen from the induced one or more male flowers; and (iii) growing a population of Cannabis sativa plants from a plurality of seed produced in (ii).

Certain aspects provide for a method of producing inbred Cannabis sativa seed comprising producing a plurality of genetically female Cannabis sativa plants with induced-male flowers, wherein the female plants comprise one or more pollen-producing fertile male flowers, produced by the method disclosed herein, wherein the female plants with induced-male flowers are grown in isolation or in coincidence with non-induced female plants of the same genotype.

Certain aspects provide for a method of producing hybrid Cannabis sativa seed comprising producing a plurality of genetically female Cannabis sativa plants with induced-male flowers, wherein the female plants comprise one or more pollen-producing fertile male flowers, produced by a method disclosed herein, wherein the female plants with induced-male flowers are grown in isolation or in coincidence with non-induced female plants having a different genotype.

Certain aspects provide for a method of screening Cannabis sativa germplasm to identify genotypes responsive to the induction of male flowers on genetically female plants comprising applying a chemical ethylene inhibitor to a genetically female plant and determining the response of the plant with respect to the induction of male flowers. In certain embodiments, the method further comprises selecting a plant based on its response to the induction of male flowers. In certain embodiments, the method further comprises genotyping the selected plant. In certain embodiments, the method further comprises identifying an association between the genotype of the selected plant and its response to the induction of male flowers. Certain aspects provide for a method of introgressing a genotype associated with responsiveness to the induction of male flowers into plant germplasm using a genotype identified by the above method. Certain aspects provide for a method of establishing inbred breeding pools of Cannabis sativa germplasm based on the genotypes identified by the above method.

Certain aspects provide for a method of processing pollen from a genetically female plant with induced-male flowers produced by a method disclosed herein, the method of pollen processing comprising: (i) removing an inflorescence-containing male flower from the female plant after mature male flowers or anthers are formed; (ii) drying the male flowers or anthers for 1 to 5 days; and then (iii) sifting to isolate pollen.

### DETAILED DESCRIPTION

To the extent necessary to provide descriptive support, the subject matter and/or text of the appended claims is incorporated herein by reference in their entirety.

Further, it will be understood by all readers of this written description that the exemplary aspects and embodiments described and claimed herein may be suitably practiced in the absence of any recited feature, element or step that is, or is not, specifically disclosed herein.

### Definitions.

It is to be noted that the term “a” or “an” entity refers to one or more of that entity; for example, “a plant” is understood to represent one or more plants. As such, the terms “a” (or “an”), “one or more,” and “at least one” can be used interchangeably herein.

Furthermore, “and/or” where used herein is to be taken as specific disclosure of each of the specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

It is understood that wherever aspects are described herein with the language “comprising” or “comprises” otherwise analogous aspects described in terms of “consisting of,” “consists of,” “consisting essentially of,” and/or “consists essentially of,” and the like are also provided.

Unless defined otherwise, technical and scientific terms used herein have the same meaning as commonly understood by one of ordinary skill in the art to which this disclosure is related.

Numeric ranges are inclusive of the numbers defining the range. Even when not explicitly identified by “and any range in between,” or the like, where a list of values is recited, e.g., 1, 2, 3, or 4, unless otherwise stated, the disclosure specifically includes any range in between the values, e.g., 1 to 3, 1 to 4, 2 to 4, etc.

For purposes of this disclosure, “flowering” in a female plant is defined by the visible formation of an inflorescence at one or more nodes.

For purposes of this disclosure, the “stem diameter” of a Cannabis sativa plant is a measurement taken 1 cm above the soil line.

Unless otherwise specified, reference to an exclusively female population of Cannabis sativa plants refers to a population of plants grown from seeds and not a clonal population of female plants.

The headings provided herein are solely for ease of reference and are not limitations of the various aspects or embodiments of the disclosure, which can be had by reference to the specification as a whole.

### Overview

Cannabis sativa has 2 copies of 10 chromosomes (2n=20) and sex is primarily determined by sex chromosomes, with XX being female and XY being male.

Cannabis sativa plants are generally grown vegetatively under long days and after transition to natural or artificial short day conditions, flowering is induced. Male plants produce staminate floral buds that open to release pollen that is naturally wind-distributed. Female plants produce flowers in a cluster, called a cola or an inflorescence. Each flower contains two stigmas and an ovule enclosed by bracts, which have the highest density of resin-containing trichomes.

### 2-aminoethoxyvinylglycine

2-aminoethoxyvinylglycine (AVG) is an inhibitor of 1-aminocyclopropane-1-carboxylic acid (ACC) synthase, a key enzyme involved in ethylene biosynthesis. In contrast, silver ions (Ag) and the gaseous compound 1-methylcyclopropene (1-MCP) have a different mode of action, and are both inhibitors of the ethylene receptors.

AVG not only inhibits ACC synthase involved in ethylene biosynthesis but also inhibits tryptophan aminotransferase involved in auxin biosynthesis, an enzyme that shares a similar pyridoxal-phosphate-dependent mechanism to ACC synthase (Schaller G. E., Binder B. M. (2017) Inhibitors of Ethylene Biosynthesis and Signaling. In: Binder B., Eric Schaller G. (eds.) Ethylene Signaling. Methods in Molecular Biology, vol. 1573. Humana Press, New York, N.Y.). In 1981, Ram et al., report delivering AVG to the apical meristem through a cotton wick to induce male flowers on the apical node of female Cannabis sativa plants, but failed to provide any evidence of the production of viable, feminized seed. In the past 30 plus years, there have been no reports of AVG being used to induce male structures or pollen, or seed produced using such pollen in Cannabis plants. Thus, given that AVG has a different mode of action on the ethylene biosynthesis pathway and that it affects the biosynthesis of another key plant hormone, auxin, it was unknown if AVG could result in conversion of female Cannabis flowers to male flowers that produced viable pollen that could be used to produce feminized seed. It was also unknown if AVG applied as a foliar spray or as a root drench would result in the movement of AVG to developing meristems to produce a robust male response required for large-scale seed production.

Feminized Cannabis sativa Seed

Provided herein are methods of producing a genetically female Cannabis sativa seed and/or embryo as well as a genetically female Cannabis sativa seed and/or embryo obtained by such methods. For the purposes of brevity, it will be understood that unless otherwise specified, reference to genetically female Cannabis sativa “seed” is shorthand encompassing genetically female Cannabis sativa “seed and/or embryo.” In certain embodiments, the genetically female Cannabis sativa seed is inbred. In certain embodiments, the genetically female Cannabis sativa seed is a hybrid. Certain embodiments provide for a Cannabis sativa plants grown from such a seed. Certain embodiments provide for a population of Cannabis sativa plants grown from a plurality of such seed. Likewise, as large Cannabis sativa operations rely on clonal propagation, certain embodiments provide for a population of exclusively female Cannabis sativa plants grown from seed. In certain embodiments, the population of Cannabis sativa plants comprises at least about 6 plants, 12 plants, 24 plants, 48 plants, 72 plants, 96 plants, 200 plants, 500 plants, 1,000 plants, 5,000 plants, 10,000 plants, 50,000 plants, 100,000 plants, 500,000 plants, or 1,000,000 plants. In certain embodiments, the population of Cannabis sativa plants comprises from any of about 6 plants, 12 plants, 24 plants, 48 plants, 72 plants, 96 plants, 200 plants, 500 plants, 1,000 plants, 5,000 plants, 10,000 plants, 50,000 plants, 100,000 plants, or 500,000 plants to any of about 12 plants, 24 plants, 48 plants, 72 plants, 96 plants, 200 plants, 500 plants, 1,000 plants, 5,000 plants, 10,000 plants, 50,000 plants, 100,000 plants, 500,000 plants or 1,000,000 plants. In certain embodiments anywhere herein, greater than 75%, 80%, 85%, 90%, 95%, 96%, 97%, 98%, 99%, 99.5%, 99.9%, or 99.99% of the plants in the population are genetically female plants. In certain embodiments anywhere herein, all of the plants in the population are genetically female plants. Certain embodiments provide for a floral bud/inflorescence of the Cannabis sativa plant disclosed above or of a Cannabis sativa plant of a population of Cannabis sativa plants disclosed above. Further, certain embodiments provide for a method of growing a Cannabis sativa plant that comprises growing a Cannabis sativa plant from a genetically female Cannabis sativa seed disclosed anywhere herein including methods of producing inbred and hybrid seeds.

In certain embodiments, the method of producing a genetically female Cannabis sativa seed comprises inducing one or more male flowers on a genetically female Cannabis sativa plant by applying a chemical ethylene inhibitor to the plant. A number of chemical ethylene inhibitors are known. Representative examples of ethylene inhibitors include aminoethoxyvinylglycine (AVG), silver thiosulfate, and 1-Methylcyclopropene (1-MCP). In certain embodiments, the ethylene inhibitor is aminoethoxyvinylglycine (AVG). Application of the chemical ethylene inhibitor causes the female plant to form one or more pollen-producing fertile male flowers. Seed can then be produced by pollinating a female flower with pollen from the induced one or more male flowers. In certain embodiments, some (<50%), most (50%-99%), or all (100%) of the seed is genetically female. In certain embodiments, the seed is produced by self-pollination of pollen from the induced one or more male flowers onto a female flower of the same plant. In certain other embodiments, the seed is produced by cross-pollination of pollen from the induced one or more male flowers onto a female flower of a separate female plant. The separate female plant used for cross-pollination can be genetically identical to the plant with the induced male flowers or the separate female plant used for cross-pollination can have a genotype that is distinct from the plant with the induced male flowers. In certain embodiments, the separate female plant used having a genotype that is distinct from the plant with the induced male flowers is from a distinct inbred strain or variety. Thus, in certain embodiments, cross-pollination creates inbred seed and in certain embodiments cross-pollination creates hybrid seed. Pollination can be natural (e.g., wind-blown), assisted, or done manually. In certain embodiments, pollination is done manually, such as by transferring the pollen to the female flower with a brush. In certain embodiments, pollination can be assisted by dispersing the pollen such as by shaking or whacking the plant and/or by creating air movement. In certain embodiments, pollen from the induced one or more male flowers is disbursed for pollination by an ultrasonic dehiscence system.

In certain embodiments, one application of the ethylene inhibitor is sufficient. It has also been discovered that multiple applications of the ethylene inhibitor can be used. In certain embodiments, the ethylene inhibitor is applied to the plant in two, three, four, five, or six separate applications. In certain embodiments, the ethylene inhibitor is applied to the plant in at least two, three, four, five, or six separate applications. In certain embodiments, the ethylene inhibitor is applied to the plant in 5 or more, 10 or more, 15 or more, or 20 or more separate applications.

Certain aspects of the methods of this disclosure involve different modes of ethylene inhibitor application. In certain embodiments, the ethylene inhibitor is applied in at least one application by root drench to the plant. In certain embodiments, the ethylene inhibitor is applied by root drench in all applications. In certain embodiments, the ethylene inhibitor is applied in at least one application by foliar spray to the plant. In certain embodiments, the ethylene inhibitor is applied by foliar spray in all applications. In certain embodiments, the ethylene inhibitor is applied in at least one application by root drench and also in at least one application by foliar spray. In certain embodiments involving at least one application by root drench and at least one application by foliar spray, the first application of the ethylene inhibitor to the plant between the two is by root drench. In certain embodiments involving at least one application by root drench and at least one application by foliar spray, the first application of the ethylene inhibitor to the plant between the two is by foliar spray.

It will be understood that in certain embodiments, certain applications of the ethylene inhibitor (e.g., AVG) comprise root drench, i.e., root drench can be in combination with application by foliar spray but does not require combination with foliar spray, while other applications comprising root drench exclude foliar spray. Likewise, certain applications comprise foliar spray, i.e., foliar spray can be in combination with application by root drench but does not require combination with root drench, while other applications comprising foliar spray exclude root drench. For example, in certain embodiments the ethylene inhibitor is applied to the plant:
(i) wherein the application comprises root drench in all applications;
(ii) wherein the application comprises root drench but not foliar spray in any applications;
(iii) wherein the application comprises foliar spray in all applications;
(iv) wherein the application comprises foliar spray but not root drench in any applications;
(v) wherein the application comprises root drench combined with foliar spray in all applications;
(vi) wherein the application comprises root drench combined with foliar spray in at least one application and root drench but not foliar spray in at least one separate application;
(vii) wherein the application comprises root drench combined with foliar spray in at least one application and foliar spray but not root drench in at least one separate application; and/or
(viii) wherein the application comprises root drench but not foliar spray in at least one application and also comprises foliar spray but not root drench in at least one separate application.

In certain embodiments, the first application of the ethylene inhibitor to the plant comprises application by root drench. In certain embodiments, the first application of the ethylene inhibitor to the plant comprises root drench application but not foliar spray. In certain embodiments, the first application of the ethylene inhibitor to the plant comprises application by foliar spray. In certain embodiments, the first application of the ethylene inhibitor to the plant comprises foliar spray application but not root drench.

Certain aspects of the methods of this disclosure involve the timing of the ethylene inhibitor application. In certain embodiments, the ethylene inhibitor is applied to the plant prior to flowering. In certain embodiments, the ethylene inhibitor is first applied to the plant prior to flowering. In certain embodiments, the ethylene inhibitor is only applied to the plant prior to flowering. In certain embodiments, the ethylene inhibitor is applied to the plant in at least two, three, four, five, or six separate applications prior to flowering. In certain embodiments, the ethylene inhibitor is applied to the plant in 5 or more, 10 or more, 15 or more, or 20 or more separate applications prior to flowering. In certain embodiments, the ethylene inhibitor is applied to the plant after flowering is initiated. In certain embodiments, the ethylene inhibitor is only applied to the plant after flowering is initiated. In certain embodiments, the ethylene inhibitor is applied to the plant in at least two, three, four, five, or six separate applications after flowering is initiated. In certain embodiments, the ethylene inhibitor is applied to the plant in 5 or more, 10 or more, 15 or more, or 20 or more separate applications after flowering is initiated. In certain embodiments, the ethylene inhibitor is re-applied 2, 3, 4, 5, 6, 7, 8, or 9 days after the initial application. Further, in certain embodiments, the ethylene inhibitor is re-applied 2, 3, 4, 5, 6, 7, 8, or 9 days after the application that is 2, 3, 4, 5, 6, 7, 8, or 9 days after the initial treatment. In certain embodiments, the ethylene inhibitor is re-applied 10, 11, 12, 13, 14, 15, 16, 17, or 18 days after the initial treatment.

In certain of any of these methods, the ethylene inhibitor can be applied to the plant prior to flowering and/or after flowering is initiated and can be applied by root drench and/or foliar spray. For example, in certain embodiments, the ethylene inhibitor is applied by root drench to the plant prior to flowering and the ethylene inhibitor is separately applied by root drench to the plant after flowering is initiated. For example, in certain embodiments, the ethylene inhibitor is applied by root drench to the plant prior to flowering and the ethylene inhibitor is separately applied by foliar spray to the plant after flowering is initiated. For example, in certain embodiments, the ethylene inhibitor is applied by foliar spray to the plant prior to flowering and the ethylene inhibitor is separately applied by root drench to the plant after flowering is initiated. For example, in certain embodiments, the ethylene inhibitor is applied by foliar spray to the plant prior to flowering and the ethylene inhibitor is separately applied by foliar spray to the plant after flowering is initiated.

As noted above, it will be understood that in certain embodiments, certain applications of the ethylene inhibitor (e.g., AVG) comprise root drench, i.e., root drench can be in combination with application by foliar spray but does not require combination with foliar spray, while other applications comprising root drench exclude foliar spray. Likewise, certain applications comprise foliar spray, i.e., foliar spray can be in combination with application by root drench but does not require combination with root drench, while other applications comprising foliar spray exclude root drench. Thus, in certain embodiments:
(i) The ethylene inhibitor application comprises root drench prior to flowering and separately comprises root drench after flowering is initiated. In certain embodiments, the ethylene inhibitor application comprises at least one root drench application without foliar spray prior to flowering. Further, in certain embodiments, the ethylene inhibitor application comprises root drench but not foliar spray prior to flowering. In certain embodiments, the ethylene inhibitor application separately comprises at least one root drench application without foliar spray after flowering is initiated. Further, in certain embodiments, the ethylene inhibitor application comprises root drench but not foliar spray after flowering is initiated;
(ii) The ethylene inhibitor application comprises root drench prior to flowering and separately comprises foliar spray after flowering is initiated. In certain embodiments, the ethylene inhibitor application comprises at least one root drench application without foliar spray prior to flowering. Further, in certain embodiments, the ethylene inhibitor application comprises root drench but not foliar spray prior to flowering. In certain embodiments, the ethylene inhibitor application separately comprises at least one foliar spray application without root drench after flowering is initiated. Further, in certain embodiments, the ethylene inhibitor application comprises foliar spray but not root drench after flowering is initiated;
(iii) The ethylene inhibitor application comprises foliar spray prior to flowering and separately comprises root drench after flowering is initiated. In certain embodiments, the ethylene inhibitor application comprises at least one foliar spray application without root drench prior to flowering. Further, in certain embodiments, the ethylene inhibitor application comprises foliar spray but not root drench prior to flowering. In certain embodiments, the ethylene inhibitor application separately comprises at least one root drench application without foliar spray after flowering is initiated. Further, in certain embodiments, the ethylene inhibitor application comprises root drench but not foliar spray after flowering is initiated; or
(iv) The ethylene inhibitor application comprises foliar spray prior to flowering and separately comprises foliar spray after flowering is initiated. In certain embodiments, the ethylene inhibitor application comprises at least one foliar spray application without root drench prior to flowering. Further, in certain embodiments, the ethylene inhibitor application comprises foliar spray but not root drench prior to flowering. In certain embodiments, the ethylene inhibitor application separately comprises at least one foliar spray application without root drench after flowering is initiated. Further, in certain embodiments, the ethylene inhibitor application comprises foliar spray and not root drench after flowering is initiated.

Certain aspects of the methods of this disclosure are drawn to inducing flowering of the plants. In certain embodiments, one the same day of or 1, 2, 3, 4, 5, 6, 7, 8, 9, 10, 11, 12, 13, 14 or 15 days after the initial application of the ethylene inhibitor to the plant, the plant is exposed to environmental conditions known to induce flowering in Cannabis sativa. In certain embodiments, one the same day of or 1, 2, 3, 4, 5, 6, 7, 8, 9, or 10 days after the initial application of the ethylene inhibitor to the plant, the plant is exposed to environmental conditions known to induce flowering in Cannabis sativa. In certain embodiments, prior to at least the initial application of the ethylene inhibitor to the plant, the plant is grown under vegetative growth conditions for Cannabis sativa. In certain embodiments, the plant is grown under vegetative growth conditions for Cannabis sativa up until 1, 2, 3, 4, 5, 6, or 7 days after the initial application of the ethylene inhibitor to the plant, at which time the plant is switched to environmental conditions that induce flowering in Cannabis sativa.

Certain aspects of the methods of this disclosure are drawn to the rate at which the ethylene inhibitor is applied. In certain embodiments, the ethylene inhibitor is applied in one or more applications by root drench in a solution containing from any of about 0.1 mg, 0.2 mg, 0.3 mg, 0.4 mg, 0.5 mg, 0.6 mg, 0.7 mg, 0.8 mg, 0.9 mg, 1.0 mg, 1.5 mg, 2.0 mg, 2.5 mg, 3.0 mg, 3.5 mg, 4.0 mg, or 5.0 mg to any of about 0.2 mg, 0.3 mg, 0.4 mg, 0.5 mg, 0.6 mg, 0.7 mg, 0.8 mg, 0.9 mg, 1.0 mg, 1.5 mg, 2.0 mg, 2.5 mg, 3.0 mg, 3.5 mg, 4.0 mg, 5.0 mg, or 10 mg of the ethylene inhibitor per millimeter of stem diameter. In certain embodiments, the ethylene inhibitor is applied at such amount in a volume of at least 25 mL, 50 mL, 100 mL, 150 mL, 200 mL, 400 mL, 500 mL, or 1 L of solution. In certain embodiments, the ethylene inhibitor is applied in one or more applications by root drench in a solution containing from any of about 0.3 mg, 0.4 mg, 0.5 mg, 0.6 mg, 0.7 mg, 0.8 mg, 0.9 mg, 1.0 mg, 1.5 mg, 2.0 mg, 2.5 mg, or 3.0 mg to any of about 0.4 mg, 0.5 mg, 0.6 mg, 0.7 mg, 0.8 mg, 0.9 mg, 1.0 mg, 1.5 mg, 2.0 mg, 2.5 mg, 3.0 mg, or 3.5 mg of the ethylene inhibitor per millimeter of stem diameter. In certain embodiments, the ethylene inhibitor is applied at such amount in a volume of at least 25 mL, 50 mL, 100 mL, 150 mL, 200 mL, 400 mL, 500 mL, or 1 L of solution. In certain embodiments, the ethylene inhibitor is applied at such amount in a volume of solution that is sufficient to come in contact of majority of root area. In certain embodiments, the volume of solution is sufficient to contact at least 50%, 60%, 70%, 80%, 90%, 95%, 98%, 99%, or 100% of the root area. In certain embodiments, the ethylene inhibitor is applied in one or more applications by root drench in a solution at a rate of any of about 5 mg/100 ml, 10 mg/100 ml, 15 mg/100 ml, 20 mg/100 ml, 25 mg/100 ml, 30 mg/100 ml, 35 mg/100 ml, 40 mg/100 ml, 45 mg/100 ml, 50 mg/100 ml, 55 mg/100 ml, 60 mg/100 ml, or 65 mg/100 ml to any of about 10 mg/100 ml, 15 mg/100 ml, 20 mg/100 ml, 25 mg/100 ml, 30 mg/100 ml, 35 mg/100 ml, 40 mg/100 ml, 45 mg/100 ml, 50 mg/100 ml, 55 mg/100 ml, 60 mg/100 ml, 65 mg/100 ml, or 75 mg/100 ml of the ethylene inhibitor in the solution. In certain embodiments, the ethylene inhibitor is applied by root drench in a solution at a rate of about 15 mg/100 ml to 50 mg/100 ml, 15 mg/100 ml to 30 mg/100 ml, or 40 mg/100 ml to 50 mg/100 ml of the ethylene inhibitor in the solution. In certain embodiments, the foliar spray is applied to apical and/or axillary nodes of the plant. In certain such embodiments, the foliar spray is applied to apical and/or axillary nodes of the plant at a rate of any of about 0.10 mg, 0.11 mg, 0.12 mg, 0.15 mg, 0.2 mg, 0.3 mg, or 0.4 mg to any of about 0.11 mg, 0.12 mg, 0.15 mg, 0.2 mg, 0.3 mg, 0.4 mg, or 0.5 mg ethylene inhibitor per ml of solution. In certain such embodiments, the foliar spray is applied to apical and/or axillary nodes of the plant at a rate of any of about 0.12 mg, 0.15 mg, 0.2 mg, or 0.3 mg to any of about 0.15 mg, 0.2 mg, 0.3 mg, or 0.4 mg ethylene inhibitor per ml of solution. In certain embodiments, the solution for foliar spray comprises 0.01% of Tween 80 solution. In certain embodiments, the above rates apply to AVG.

Certain aspects of the methods of this disclosure result in the production of Cannabis sativa seed, and in certain embodiments, large numbers, such on a production and/or commercial scale. In certain embodiments, at least about 5, 10, 25, 50, 100, 125, 150, 200, 250, 500, 750, 1,000, 1,250, 1,500, 2,000, 3,000, 4,000, or 5,000 genetically female Cannabis sativa seeds are produced on a self-pollinated plant. In certain embodiments, any of about 5, 10, 25, 50, 100, 125, 150, 200, 250, 500, 750, 1,000, 1,250, 1,500, 2,000, 3,000, or 4,000, to any of about 10, 25, 50, 100, 125, 150, 200, 250, 500, 750, 1,000, 1,250, 1,500, 2,000, 3,000, 4,000, or 5,000 genetically female Cannabis sativa seeds are produced on a self-pollinated plant. In certain embodiments, all the seeds produced on the self-pollinated plant are genetically female. In certain embodiments, at least about 5, 10, 25, 50, 100, 125, 150, 200, 250, 500, 750, 1,000, 1,250, 1,500, 2,000, 3,000, 4,000, or 5,000 genetically female Cannabis sativa seeds are produced on a cross-pollinated plant. In certain embodiments, any of about 5, 10, 25, 50, 100, 125, 150, 200, 250, 500, 750, 1,000, 1,250, 1,500, 2,000, 3,000, or 4,000 to any of about 10, 25, 50, 100, 125, 150, 200, 250, 500, 750, 1,000, 1,250, 1,500, 2,000, 3,000, 4,000, or 5,000 genetically female Cannabis sativa seeds are produced on a cross-pollinated plant. In certain embodiments, greater than about 75%, 80%, 85%, 90%, 95%, 96%, 97%, 98%, 99%, 99.9% or 99.99% of the seeds produced on a cross-pollinated plant are genetically female. In certain embodiments, all the seeds produced on the cross-pollinated plant are genetically female. When the method is applied to a plurality or population of plants, genetically female Cannabis sativa seed can be produced on a production and/or commercial scale. In certain embodiments, any of about 5,000, 10,000, 20,000, 50,000, 100,000, or 500,000 to any of about 10,000, 20,000, 50,000, 100,000, 500,000, or 1,000,000 genetically female Cannabis sativa seeds are produced. In certain embodiments, at least about 5,000, 10,000, 20,000, 50,000, 100,000, 500,000, or 1,000,000 genetically female Cannabis sativa seeds are produced. In certain embodiments, the seeds produced are exclusively genetically female. In certain embodiments, the genetically female seed can be packaged into bags, containers, seed lots, commercial seed lots, and the like having any of about 5,000, 10,000, 20,000, 50,000, 100,000, or 500,000, to any of about 10,000, 20,000, 50,000, 100,000, 500,000 or 1,000,000 genetically female Cannabis sativa seeds wherein greater than about 75%, 80%, 85%, 90%, 95%, 96%, 97%, 98%, 99%, 99.9% or 99.99% of the seeds in the bags, containers, seed lots, commercial seed lots and the like are genetically female Cannabis sativa seeds. In certain embodiments, the genetically female seed can be packaged into bags, containers, seed lots, commercial seed lots, and the like having at least 5,000, 10,000, 20,000, 50,000, 100,000, 500,000, or 1,000,000 exclusively genetically female Cannabis sativa seeds. In certain embodiments, the genetically female seed can be packaged into bags, containers, seed lots, commercial seed lots, and the like having any of about 5,000, 10,000, 20,000, 50,000, 100,000, or 500,000, to any of about 10,000, 20,000, 50,000, 100,000, 500,000 or 1,000,000 genetically female Cannabis sativa seeds wherein greater than about 75%, 80%, 85%, 90%, 95%, 96%, 97%, 98%, 99%, 99.9% or 99.99% of the seeds in the bags, containers, seed lots, commercial seed lots and the like are genetically female Cannabis sativa seeds. In certain embodiments, the genetically female seed can be packaged into bags, containers, seed lots, commercial seed lots, and the like having any of about 5,000, 10,000, 20,000, 50,000, 100,000, or 500,000, to any of about 10,000, 20,000, 50,000, 100,000, 500,000 or 1,000,000 exclusively genetically female Cannabis sativa seeds. Thus, certain embodiments provide for a method of packaging bags, containers, seed lots, and/or commercial seed lots according to the above and for bags, containers, seed lots, and/or commercial seed lots of seeds according to the above. Certain embodiments provide for methods including the steps of producing and further packaging genetically female Cannabis sativa seed incorporating the methods of producing genetically female Cannabis sativa seed disclose anywhere herein.

As noted, the genetically female Cannabis sativa seed produced by the methods of this disclosure can be grown into Cannabis sativa plants. Thus, certain embodiments comprise or further comprise growing a Cannabis sativa plant from the genetically female Cannabis sativa seed produced. It will be understood that the party generating the feminized seed can further grow that seed, or, they can transfer the seed they produce to another party to grow the seed. Both are specifically contemplated in this disclosure. Certain embodiments comprise growing from a plurality of seeds a population of Cannabis sativa plants. The population may be small, such as two or three or four or five plants, such as for personal use or the population may be large, such as on a commercial scale, such as hundreds, or thousands, or tens of thousands, or hundreds of thousands, or millions of genetically female plants. For example, in certain embodiments, the population comprises at least about 6 plants, 12 plants, 24 plants, 48 plants, 72 plants, 96 plants, 200 plants, 500 plants, 1,000 plants, 5,000 plants, 10,000 plants, 50,000 plants, or 100,000 genetically female plants. In certain embodiments, the population has greater than about 75%, 80%, 85%, 90%, 95%, 96%, 97%, 98%, 99%, 99.9% or 99.99% genetically female plants. In certain embodiments, the population is exclusively composed of genetically female.

The Cannabis sativa plant can be grown for its floral bud/inflorescence which is generally considered the main consumable portion of the plant. Certain embodiments are drawn to a floral bud/inflorescence of a plant grown from a seed produced by a method of this disclosure.

Certain embodiments are drawn to a seed of a Cannabis sativa plant grown from a seed produced by a method of this disclosure. In certain embodiments, the seed is part of a plurality of seeds and/or a seed lot such as a commercial seed lot. Certain embodiments are drawn to a Cannabis sativa plant grown from such a seed or a progeny thereof. Certain embodiments are drawn to a population of Cannabis sativa plants grown from a plurality of such seed. In certain embodiments, the population comprises at least about 6 plants, 12 plants, 24 plants, 48 plants, 72 plants, 96 plants, 200 plants, 500 plants, 1,000 plants, 5,000 plants, 10,000 plants, 50,000 plants, or 100,000 plants. In certain embodiments, greater than about 75%, 80%, 85%, 90%, 95%, 96%, 97%, 98%, 99%, 99.9% or 99.99% of the plants in the population are genetically female plants. In certain embodiments, all of the plants in the population are genetically female plants.

Also provided for herein are methods of inducing male flowers on a genetically female Cannabis sativa plant by applying a chemical ethylene inhibitor (as described elsewhere herein) to a genetically female Cannabis sativa plant as well as a genetically female Cannabis sativa plant comprising induced fertile male flowers produced by such methods. In certain embodiments, the chemical ethylene inhibitor is aminoethoxyvinylglycine (AVG). In certain embodiments, the female plant forms one or more pollen-producing fertile male flowers. Consistent with methods described in greater detail elsewhere herein (which are incorporate in the methods of inducing male flowers), in certain embodiments, one application of the ethylene inhibitor is sufficient. And, in certain embodiments, the ethylene inhibitor is applied to the plant multiple time, e.g., in at least two, three, four, five, or six separate applications. In certain embodiments, the ethylene inhibitor is applied to the plant in 5 or more, 10 or more, 15 or more, or 20 or more separate applications.

In certain embodiments, the ethylene inhibitor is applied in at least one application by root drench to the plant. In certain embodiments, the ethylene inhibitor is applied in at least one application by foliar spray to the plant. In certain embodiments, the ethylene inhibitor is applied to the plant: by root drench in all applications; by foliar spray in all applications; or by root drench in at least one application and also by foliar spray in at least one separate application. In certain embodiments where the ethylene inhibitor is applied by root drench in at least one application and also by foliar spray in at least one separate application; the first application between the two of the ethylene inhibitor to the plant is by root drench. In certain embodiments where the ethylene inhibitor is applied by root drench in at least one application and also by foliar spray in at least one separate application; the first application between the two of the ethylene inhibitor to the plant is by foliar spray.

It will be understood that in certain embodiments, certain applications of the ethylene inhibitor (e.g., AVG) comprise root drench, i.e., root drench can be in combination with application by foliar spray but does not require combination with foliar spray, while other applications comprising root drench exclude foliar spray. Likewise, certain applications comprise foliar spray, i.e., foliar spray can be in combination with application by root drench but does not require combination with root drench, while other applications comprising foliar spray exclude root drench. For example, in certain embodiments the ethylene inhibitor is applied to the plant:
(i) wherein the application comprises root drench in all applications;
(ii) wherein the application comprises root drench but not foliar spray in any applications;
(iii) wherein the application comprises foliar spray in all applications;
(iv) wherein the application comprises foliar spray but not root drench in any applications;
(v) wherein the application comprises root drench combined with foliar spray in all applications;
(vi) wherein the application comprises root drench combined with foliar spray in at least one application and root drench but not foliar spray in at least one separate application;
(vii) wherein the application comprises root drench combined with foliar spray in at least one application and foliar spray but not root drench in at least one separate application; and/or
(viii) wherein the application comprises root drench but not foliar spray in at least one application and also comprises foliar spray but not root drench in at least one separate application.

In certain embodiments, the first application of the ethylene inhibitor to the plant comprises application by root drench. In certain embodiments, the first application of the ethylene inhibitor to the plant comprises root drench application but not foliar spray. In certain embodiments, the first application of the ethylene inhibitor to the plant comprises application by foliar spray. In certain embodiments, the first application of the ethylene inhibitor to the plant comprises foliar spray application but not root drench.

Certain aspects of the methods of this disclosure involve the timing of the ethylene inhibitor application. In certain embodiments, the ethylene inhibitor is applied to the plant prior to flowering. In certain embodiments, the ethylene inhibitor is first applied to the plant prior to flowering. In certain embodiments, the ethylene inhibitor is only applied to the plant prior to flowering. In certain embodiments, the ethylene inhibitor is applied to the plant in at least two, three, four, five, or six separate applications prior to flowering. In certain embodiments, the ethylene inhibitor is applied to the plant in 5 or more, 10 or more, 15 or more, or 20 or more separate applications prior to flowering. In certain embodiments, the ethylene inhibitor is applied to the plant after flowering is initiated. In certain embodiments, the ethylene inhibitor is only applied to the plant after flowering is initiated. In certain embodiments, the ethylene inhibitor is applied to the plant in at least two, three, four, five, or six separate applications after flowering is initiated. In certain embodiments, the ethylene inhibitor is applied to the plant in 5 or more, 10 or more, 15 or more, or 20 or more separate applications after flowering is initiated. In certain embodiments, the ethylene inhibitor is re-applied 2, 3, 4, 5, 6, 7, 8, or 9 days after the initial application. Further, in certain embodiments, the ethylene inhibitor is re-applied 2, 3, 4, 5, 6, 7, 8, or 9 days after the application that is 2, 3, 4, 5, 6, 7, 8, or 9 days after the initial treatment. In certain embodiments, the ethylene inhibitor is re-applied 10, 11, 12, 13, 14, 15, 16, 17, or 18 days after the initial treatment.

In certain of any of these methods, the ethylene inhibitor can be applied to the plant prior to flowering and/or after flowering is initiated and can be applied by root drench and/or foliar spray. For example, in certain embodiments, the ethylene inhibitor is applied by root drench to the plant prior to flowering and the ethylene inhibitor is separately applied by root drench to the plant after flowering is initiated. For example, in certain embodiments, the ethylene inhibitor is applied by root drench to the plant prior to flowering and the ethylene inhibitor is separately applied by foliar spray to the plant after flowering is initiated. For example, in certain embodiments, the ethylene inhibitor is applied by foliar spray to the plant prior to flowering and the ethylene inhibitor is separately applied by root drench to the plant after flowering is initiated. For example, in certain embodiments, the ethylene inhibitor is applied by foliar spray to the plant prior to flowering and the ethylene inhibitor is separately applied by foliar spray to the plant after flowering is initiated.

As noted above, it will be understood that in certain embodiments, certain applications of the ethylene inhibitor (e.g., AVG) comprise root drench, i.e., root drench can be in combination with application by foliar spray but does not require combination with foliar spray, while other applications comprising root drench exclude foliar spray. Likewise, certain applications comprise foliar spray, i.e., foliar spray can be in combination with application by root drench but does not require combination with root drench, while other applications comprising foliar spray exclude root drench. Thus, in certain embodiments:
(i) The ethylene inhibitor application comprises root drench prior to flowering and separately comprises root drench after flowering is initiated. In certain embodiments, the ethylene inhibitor application comprises at least one root drench application without foliar spray prior to flowering. Further, in certain embodiments, the ethylene inhibitor application comprises root drench but not foliar spray prior to flowering. In certain embodiments, the ethylene inhibitor application separately comprises at least one root drench application without foliar spray after flowering is initiated. Further, in certain embodiments, the ethylene inhibitor application comprises root drench but not foliar spray after flowering is initiated;
(ii) The ethylene inhibitor application comprises root drench prior to flowering and separately comprises foliar spray after flowering is initiated. In certain embodiments, the ethylene inhibitor application comprises at least one root drench application without foliar spray prior to flowering. Further, in certain embodiments, the ethylene inhibitor application comprises root drench but not foliar spray prior to flowering. In certain embodiments, the ethylene inhibitor application separately comprises at least one foliar spray application without root drench after flowering is initiated. Further, in certain embodiments, the ethylene inhibitor application comprises foliar spray but not root drench after flowering is initiated;
(iii) The ethylene inhibitor application comprises foliar spray prior to flowering and separately comprises root drench after flowering is initiated. In certain embodiments, the ethylene inhibitor application comprises at least one foliar spray application without root drench prior to flowering. Further, in certain embodiments, the ethylene inhibitor application comprises foliar spray but not root drench prior to flowering. In certain embodiments, the ethylene inhibitor application separately comprises at least one root drench application without foliar spray after flowering is initiated. Further, in certain embodiments, the ethylene inhibitor application comprises root drench but not foliar spray after flowering is initiated; or
(iv) The ethylene inhibitor application comprises foliar spray prior to flowering and separately comprises foliar spray after flowering is initiated. In certain embodiments, the ethylene inhibitor application comprises at least one foliar spray application without root drench prior to flowering. Further, in certain embodiments, the ethylene inhibitor application comprises foliar spray but not root drench prior to flowering. In certain embodiments, the ethylene inhibitor application separately comprises at least one foliar spray application without root drench after flowering is initiated. Further, in certain embodiments, the ethylene inhibitor application comprises foliar spray and not root drench after flowering is initiated.

In certain embodiments, the ethylene inhibitor is re-applied 2, 3, 4, 5, 6, 7, 8, or 9 days after the initial treatment. In certain embodiments, the ethylene inhibitor is further re-applied 2, 3, 4, 5, 6, 7, 8, or 9 days after the application that is 2, 3, 4, 5, 6, 7, 8, or 9 days after the initial treatment. In certain embodiments, the ethylene inhibitor is re-applied 10, 11, 12, 13, 14, 15, 16, 17, or 18 days after the initial treatment. In certain embodiments, on the same day of or 1, 2, 3, 4, 5, 6, 7, 8, 9, 10, 11, 12, 13, 14 or 15 days after the initial application of the ethylene inhibitor to the plant, the plant is exposed to environmental conditions that induce flowering in Cannabis sativa. In certain embodiments, on the same day of or 1, 2, 3, 4, 5, 6, 7, 8, 9, or 10 days after the initial application of the ethylene inhibitor to the plant, the plant is exposed to environmental conditions that induce flowering in Cannabis sativa. In certain embodiments, prior to the initial application of the ethylene inhibitor to the plant, the plant is grown under vegetative growth conditions for Cannabis sativa. In certain embodiments, the plant is grown under vegetative growth conditions for Cannabis sativa up until 1, 2, 3, 4, 5, 6, or 7 days after the initial application of the ethylene inhibitor to the plant, at which time the plant is switched to environmental conditions that induce flowering in Cannabis sativa. In certain embodiments, the ethylene inhibitor is applied by root drench and/or foliar spray at a rate described in detail elsewhere herein.

Certain aspects of this disclosure are drawn to methods of producing an inbred variety of Cannabis sativa. In certain embodiments, the method comprises first producing a genetically female Cannabis sativa plant with induced-male flowers, wherein the female plant comprises one or more pollen-producing fertile male flowers, by any of the methods described elsewhere herein. The method next comprises producing seed by self-pollination of pollen from an induced-male flower onto a female flower of the same plant or by cross-pollination of pollen from an induced-male flower onto a female flower of a separate but genetically identical female plant. In certain embodiments, the genetically identical female plant is produced through clonal propagation. Certain embodiments comprise growing a genetically female Cannabis sativa plant from a seed produced above. In certain embodiments, these steps can be repeated, for example for at least two, three, four, five, six, or seven or more generations. Certain embodiments provide for an inbred Cannabis sativa seed produced by such methods. In certain embodiments, the seed is part of a plurality of seeds and/or seed lot, such as a commercial seed lot and/or comprising a number of seeds and/or seed composition as disclosed elsewhere herein. Certain embodiments provide for an inbred variety of Cannabis sativa, or a plant of the variety, produced by such methods of producing an inbred variety of Cannabis sativa. Certain embodiments provide for a population of inbred Cannabis sativa plants comprising an inbred variety produced by such methods of producing an inbred variety of Cannabis sativa. In certain embodiments, the population of inbred Cannabis sativa plants comprises at least about 6 plants, 12 plants, 24 plants, 48 plants, 72 plants, 96 plants, 200 plants, 500 plants, 1,000 plants, 5,000 plants, 10,000 plants, 50,000 plants, or 100,000 plants. In certain embodiments, the population of inbred Cannabis sativa plants comprises from any of about 6 plants, 12 plants, 24 plants, 48 plants, 72 plants, 96 plants, 200 plants, 500 plants, 1,000 plants, 5,000 plants, 10,000 plants, or 50,000 plants to any of about 12 plants, 24 plants, 48 plants, 72 plants, 96 plants, 200 plants, 500 plants, 1,000 plants, 5,000 plants, 10,000 plants, 50,000 plants, or 100,000 plants. In certain embodiments of a population of inbred Cannabis sativa plants, all of the plants in the population are genetically female plants. Certain embodiments provide for a floral bud/inflorescence of an inbred Cannabis sativa plant or of an inbred Cannabis sativa plant of a population of plants described above. Certain embodiments provide for a seed from the inbred Cannabis sativa variety or inbred plant described above. The seed could be inbred or could be the result of hybridization. In certain embodiments, the seed is part of a plurality of seeds and/or seed lot as described elsewhere herein.

Certain embodiments are also drawn to methods of producing reproducible hybrid Cannabis sativa seed comprising crossing two distinct inbred Cannabis sativa varieties, wherein at least one inbred variety has been produced by a method of producing an inbred variety disclosed herein. In certain embodiments, both inbred varieties have been produced by a method of producing an inbred variety disclosed herein. Certain embodiments also provide for a hybrid Cannabis sativa seed produced by a method of producing an inbred variety disclosed herein. In certain embodiments, the seed is part of a plurality of seeds and/or seed lot as described elsewhere herein. Certain embodiments also provide for a hybrid Cannabis sativa plant grown from such seed and also a population of hybrid Cannabis sativa plants grown from a plurality such seeds. In certain embodiments, the population of hybrid Cannabis sativa plants comprises at least about 6 plants, 12 plants, 24 plants, 48 plants, 72 plants, 96 plants, 200 plants, 500 plants, 1,000 plants, 5,000 plants, 10,000 plants, 50,000 plants, or 100,000 plants. In certain embodiments, the population of hybrid Cannabis sativa plants comprises from any of about 6 plants, 12 plants, 24 plants, 48 plants, 72 plants, 96 plants, 200 plants, 500 plants, 1,000 plants, 5,000 plants, 10,000 plants, or 50,000 plants to any of about 12 plants, 24 plants, 48 plants, 72 plants, 96 plants, 200 plants, 500 plants, 1,000 plants, 5,000 plants, 10,000 plants, 50,000 plants, or 100,000 plants. In certain embodiments of a population of hybrid Cannabis sativa plants, all of the plants in the population are genetically female plants. Certain embodiments provide for a floral bud/inflorescence of a hybrid Cannabis sativa plant or of a hybrid Cannabis sativa plant of a population of plants described above.

Certain aspects of methods of this disclosure provide for producing a population of Cannabis sativa plants. In certain embodiments, the method comprises the steps of: i) producing a genetically female Cannabis sativa plant with induced-male flowers, wherein the female plant comprises one or more pollen-producing fertile male flowers, as described in detail elsewhere herein; (ii) producing seed by pollinating a female flower with pollen from the induced one or more male flowers; and (iii) growing a population of Cannabis sativa plants from a plurality of seed produced in (ii). In certain embodiments, the seed produced in (ii) is produced by self-pollination of pollen from the induced one or more male flowers onto a female flower of the same plant. In certain embodiments, the seed produced in (ii) is produced by cross-pollination of pollen from the induced one or more male flowers onto a female flower of a separate female plant. In certain embodiments, the separate female plant used for cross-pollination is genetically identical to the plant with the induced male flowers. In certain embodiments, the separate female plant used for cross-pollination that is genetically identical is a clone of the plant with the induced flowers. In certain other embodiments, the separate female plant used for cross-pollination has a genotype that is distinct from the plant with the induced male flowers. In certain embodiments, the separate female plant used having a genotype that is distinct from the plant with the induced male flowers is from a distinct inbred variety. In certain embodiments, the population comprises at least about 6 plants, 12 plants, 24 plants, 48 plants, 72 plants, 96 plants, 200 plants, 500 plants, 1,000 plants, 5,000 plants, 10,000 plants, 50,000 plants, 100,000 plants, or 500,000. In certain embodiments, the population comprises from any of about 6 plants, 12 plants, 24 plants, 48 plants, 72 plants, 96 plants, 200 plants, 500 plants, 1,000 plants, 5,000 plants, 10,000 plants, 50,000 plants, 100,000, or 500,000 plants to any of about 12 plants, 24 plants, 48 plants, 72 plants, 96 plants, 200 plants, 500 plants, 1,000 plants, 5,000 plants, 10,000 plants, 50,000 plants, 100,000, 500,000 plants, or 1,000,000 plants. In certain embodiments, at least about 75%, 80%, 85%, 90%, 95%, 96%, 97%, 98%, 99%, 99.9% or 99.99% of the plants in the population are genetically female plants. In certain embodiments, all of the plants in the population are genetically female plants.

Certain aspects of this disclosure provide for producing inbred Cannabis sativa seed by producing one or a plurality of genetically female Cannabis sativa plants with induced-male flowers by the methods described in detail elsewhere herein, wherein the female plant(s) comprise one or more pollen-producing fertile male flowers. In certain embodiments, the female plant(s) with induced-male flowers are grown in isolation or in coincidence with non-induced female plants of the same genotype. In certain embodiments, the method is used to produce seed on a commercial or production scale. For example, in certain embodiments, the method is used to produce at least about 100 inbred seeds, 500 inbred seeds, 1,000 inbred seeds, 5,000 inbred seeds, 10,000 inbred seeds, 50,000 inbred seeds, 100,000 inbred seed, 500,000 inbred seeds, 1,000,000 inbred seeds. In certain embodiments, the method is used to produce any of about 100 inbred seeds, 500 inbred seeds, 1,000 inbred seeds, 5,000 inbred seeds, 10,000 inbred seeds, 50,000 inbred seeds, 100,000 inbred seed, or 500,000 inbred seeds to any of about 500 inbred seeds, 1,000 inbred seeds, 5,000 inbred seeds, 10,000 inbred seeds, 50,000 inbred seeds, 100,000 inbred seed, 500,000 inbred seeds, or 1,000,000 inbred seeds. Pollen generated from the induced-male flowers can be used in a variety of ways. In certain embodiments, the pollen from the induced-male flowers naturally dehiscesses. In certain embodiments, the pollen from the induced-male flowers is collected for immediate application to female flowers. In certain embodiments, the pollen from the induced-male flowers is stored for later application. And, in certain embodiments, the pollen from the induced-male flowers is disbursed for pollination by an ultrasonic dehiscence system as described elsewhere herein.

Certain aspects of this disclosure provide for producing hybrid Cannabis sativa seed by producing one or a plurality of genetically female Cannabis sativa plant(s) with induced-male flowers as described in detail elsewhere herein, wherein the female plant(s) comprise one or more pollen-producing fertile male flowers. In certain embodiments, the female plant(s) with induced-male flowers are grown in isolation or in coincidence with non-induced female plants having a different genotype. In certain embodiments, the non-induced female plant having a different genotype is an inbred plant. In certain embodiments, the hybrid seed is produced on a production or commercial scale. For example, in certain embodiments, the method is used to produce at least about 100 hybrid seeds, 500 hybrid seeds, 1,000 hybrid seeds, 5,000 hybrid seeds, 10,000 hybrid seeds, 50,000 hybrid seeds, 100,000 hybrid seed, 500,000 hybrid seeds, 1,000,000 hybrid seeds. In certain embodiments, the method is used to produce any of about 100 hybrid seeds, 500 hybrid seeds, 1,000 hybrid seeds, 5,000 hybrid seeds, 10,000 hybrid seeds, 50,000 hybrid seeds, 100,000 hybrid seed, or 500,000 hybrid seeds to any of about 500 hybrid seeds, 1,000 hybrid seeds, 5,000 hybrid seeds, 10,000 hybrid seeds, 50,000 hybrid seeds, 100,000 hybrid seed, 500,000 hybrid seeds, or 1,000,000 hybrid seeds. In certain embodiments, the pollen from the induced-male flowers naturally dehiscesses. In certain embodiments, the pollen from the induced-male flowers is collected for immediate application to female flowers. In certain embodiments, the pollen from the induced-male flowers is stored for later application. And, in certain embodiments, the pollen from the induced-male flowers is disbursed for pollination by an ultrasonic dehiscence system as described elsewhere herein.

Certain aspects of this disclosure also provide for screening Cannabis sativa germplasm to identify genotypes responsive to the induction of male flowers on genetically female plants by applying a chemical ethylene inhibitor (e.g., AVG) to a genetically female plant and determining the response of the plant with respect to the induction of male flowers. In certain embodiments, the induction of male flowers is done as described in detail elsewhere herein. In certain embodiments, a plant is selected based on its response to the induction of male flowers. In certain embodiments, one or more plants is genotyped. In certain embodiments, a plant selected based on its response to the induction of male flowers is genotyped and, or otherwise, in certain embodiments an association between the genotype of the selected plant and its response to the induction of male flowers is identified. In certain embodiments, the genotype is associated with responsiveness to the induction of male flowers such as the production of pollen-producing fertile male flowers. In certain embodiments, the genotype is associated with a lack of responsiveness to the induction of male flowers. One of ordinary skill in the art will recognize that in any of these method and/or embodiments, a plant is selected for use, treatment, pollination, crossing, etc. based on knowledge of its genotype. One of ordinary skill in the art will also recognize that the steps of applying the inhibitor, genotyping, selecting plants, and/or identifying an association can be performed by a single party or may be performed by multiple parties, such as those working together to identify genotypes responsive to the induction of male flowers. Further, in certain embodiments, a genetically female Cannabis sativa plant comprising a genotype associated with responsiveness to the induction of male flowers is treated to induce male flowers and/or produce seed as described elsewhere herein. In certain embodiments, a Cannabis sativa plant comprising a genotype associated with responsiveness to the induction of male flowers is self-pollinated. In certain embodiments, a Cannabis sativa plant comprising a genotype associated with responsiveness to the induction of male flowers is crossed to a second Cannabis sativa plant. In certain embodiments, the second Cannabis sativa plant does not comprise the same genotype associated with responsiveness to the induction of male flowers as the first plant. In certain embodiments, a population of Cannabis sativa plants segregating for a genotype associated with responsiveness to the induction of male flowers is generated. Further provided for is the introgression into the genome of a Cannabis sativa plant of a genotype associated with responsiveness to the induction of male flowers into plant germplasm using a genotype identified as associated with responsiveness to the induction of male flowers.

Certain aspects of this disclosure provide for a method of establishing inbred breeding pools of Cannabis sativa germplasm based on the genotypes identified by the methods disclosed herein. In certain embodiments, those genotypes are associated with responsiveness to the induction of male flowers on female plants. In certain embodiments, those genotypes are associated with a lack of responsiveness to the induction of male flowers on female plants. Once established, such breeding pools can be used, for example, in hybrid Cannabis sativa plant development.

Certain aspects provide for a method of processing pollen from a genetically female plant with induced-male flowers produced as described elsewhere herein. In certain embodiments, the method comprises removing an inflorescence-containing male flower from the female plant after mature male flowers or anthers are formed and drying the male flowers or anthers for about 1, 2, 3, 4, 5, 6, or 7 days. The dried male flowers or anthers are then sifted to isolate pollen. The pollen can then be used immediately or it can be storage for later use.

Representative, non-limiting strains of Cannabis sativa on which male-flower induction and seed production may occur include those referred to in the field as Wily Jack, Birthday Cake, Mimosa, Gorilla Glue, Capstone, Sour Strawberry, Strawanna, and BioDiesel.

### EXAMPLES

### Example 1

### Ethylene Inhibitor Treatment of Cannabis sativa Plants

Two replicate clones of five separate strains/genotypes were selected to test the effects of multiple treatments of 2-aminoethoxyvinylglycine (AVG) prior to initiation of flowering. Clones were transplanted into 4 L pots with peat-based substrate. The plants were grown outside under natural long day conditions (16 hour day) for 39-49 days (depending on strain), when they received their first AVG treatment. The stems of the plants ranged from 9-11 mm at the time of treatment, which was correlated with plant size and vigor. AVG was applied as a root drench by pouring 200 ml of AVG solution (AVG in water) near the base of the plant (aka “root drench”). Strains A and B had more biomass and each plant was treated with 100 mg of the product ReTain, which contains 15% AVG (15 mg AVG), which was dissolved in 200 ml of water. Strains C, D and E were smaller and treated with 12 mg AVG dissolved in 200 ml of water (Table 1).

TABLE 1
Plant size and treatment rates.

| | | Plant | | Plant | | Stem | | |
|---|---|---|---|---|---|---|---|---|
| | Plant | Height | | Width | | Diameter | | |
| | Archi- | (cm) | | (cm) | | (mm) | | AVG |
| Strain | tecture | Rep 1 | Rep 2 | Rep 1 | Rep 2 | Rep 1 | Rep 2 | mg/vol |
| A | tall and | 77.5 | 74.5 | 41 | 38 | 11 | 10 | 15 mg/ |
| | lanky | | | | | | | 200 ml |
| B | medium, | 59 | 55.5 | 30 | 35 | 9 | 10 | 15 mg/ |
| | branching | | | | | | | 200 ml |
| C | small, | 46.5 | 42 | 18 | 20 | 9 | 9 | 12 mg/ |
| | compact | | | | | | | 200 ml |
| D | smaller, | 44 | 37.5 | 33 | 29 | 10 | 9 | 12 mg/ |
| | compact | | | | | | | 200 ml |
| E | smaller, | 37.5 | 36.6 | 30 | 29 | 9 | 9 | 12 mg/ |
| | compact | | | | | | | 200 ml |

Five days after the first AVG treatment, the plants were transferred to a controlled environment with a 13 h day and an 11 h night, to induce flowering. The initiation of flowering was observed 4 days after the transition to short days (13 h). Seven and 14 days after the first treatment, the same AVG treatment, as above, was applied, for a total of three treatments. By three days after the first AVG treatment, the apical and axillary meristems showed signs of chlorosis, indicating the AVG chemistry was successfully translocated through the roots into aerial parts of the plants. The amount of chlorosis observed was strain dependent, with strain A showing the strongest chlorosis, strain B showing mild chlorosis and strains C, D and E showing intermediate chlorosis. All plants grew out of the chlorosis, and plant health was not impacted.

### Example 2

### Production of Male Structures, Pollen Collection, and Seed Harvest

Strains A, C, D, and E started producing chemically induced male flowers 14-22 days after being transferred to short days to induce flowering, which was 19-27 days after the first AVG treatment. Strain B did not develop chemically induced male flowers. Overall, strain A produced the most chemically induced male flowers, with large inflorescences being exclusively male, although female structures were formed on other branches, but always with male structures, as well. Apical portions of branches were more highly converted toward maleness, while interior portions were more likely to remain partially female. High quality pollen was produced from male flowers over the course of a week. Strain D was the most productive, with high quality pollen produced over 15 days. Strain C produced pollen over 11 days. Strain E produced a small amount of pollen over 3 days. Strain B never produced pollen.

To obtain pollen for controlled pollinations, extruded anthers or whole male flowers were collected using fine forceps. These tissues were then 1) immediately gently sieved with a 355 micron sieve or 2) allowed to dry for approximately 24 h, 36 h, or 48 h, and then sieved to release pollen. Pollen quality was assessed under a microscope (10× magnification). Clear, round pollen was scored as high quality. Clear, angular pollen was scored as moderate quality. Angular and cloudy pollen was scored as low quality. Pollen was applied to female flowers using a small paintbrush.

To achieve efficient, whole plant pollinations, since chemically induced male flowers do not dehisce as readily as natural males, an ultrasonic dehiscence system was used to mobilize clouds of airborne pollen from chemically induced male flowers onto an adjacent female plant for pollination. Ultrasonic devices (˜100-500 Hz) were applied directly to chemically induced male inflorescences, branch stems or the main stem to mobilize pollen from mature male flowers.

Approximately 30-45 days after pollination, individual inflorescences, or stems containing multiple inflorescences, as appropriate, were removed from the plant and placed in a fine mesh bag and allowed to fully dry out for at least 7 days. After this desiccation step, the seeds were removed from the dried inflorescences through manual manipulation. Seeds were then counted and placed in seed tubes for storage.

Alternatively, approximately 30-45 days after pollination, the plants were allowed to dry out fully in their pots for about 3 days. Inflorescences or branches with marked controlled crosses were collected individually, allowed to dry at approximately 75-80 degrees Fahrenheit for at least 7 days, then seeds were harvested, counted and stored. Remaining plants or branches were removed and dried similarly, and seed produced through ultrasonic vibration pollination was harvested, counted and stored.

### Example 3

### Production of Cannabis sativa Selfed Seed Through Controlled Pollinations

Because female flowers and chemically induced male flowers were present on responsive strains, it was possible to create selfed seed. To obtain a small quantity of selfed seed, pollen was collected from chemically induced male structures (yellow stamen and whole male flowers) of Strain D, plant rep number 1 (D1). This pollen was used to immediately pollinate female inflorescences on the same plant, D1, as described in Example 2. Some harvested chemically induced male structures were allowed to dry for 24 h at room temperature (70-79 degrees Fahrenheit), after which they were sieved to release and collect pollen. The pollen was applied to female inflorescences on the genetically identical clone, plant D2, demonstrating an alternative method to obtain the equivalent of selfed seed. Seed counts are reported in Table 2.

TABLE 2
Summary of self-crosses and resulting seed counts.

| CROSS | Pollination | Female × | | Seed |
|---|---|---|---|---|
| # | Type | Induced Male | Comment | Count |
| 21 | Self | E2 × E2 | 24 h dried anthers, one small | 3 |
| | | | inflorescence pollinated | |
| 40 | Self | D1 × D1 | Fresh pollen | 44 |
| 42 | Sib Cross | D2 × D1 | 24 h dried anthers | 35 |
| 43 | Sib Cross | D2 × D1 | 24 h dried anthers | 20 |
| 44 | Sib Cross | D2 × D1 | Fresh pollen used on multiple | 48 |
| | | | small inflorescences | |
| 45 | Sib Cross | D2 × D1 | 36 h dried anthers used on | 63 |
| | | | multiple inflorescences | |
| 46 | Sib Cross | A1 × A2 | Fresh pollen, but small amount | 19 |
| 48 | Sib Cross | A1 × A2 | 24 h dried anthers, good | 37 |
| | | | amount of pollen | |
| 49 | Sib Cross | A1 × A2 | 36 h dried anthers | 8 |
| 50 | Sib Cross | A1× A2 | 24 h dried anthers, 2 small | 4 |
| | | | flowers pollinated | |
| 51 | Self | C2 × C2 | 20 h dried anthers, moderate | 3 |
| | | | quality pollen grains, but good | |
| | | | amount | |

Similarly, male structures (yellow stamen and whole male flowers) were harvested from induced male inflorescences from Strain C, plant rep number 2 (C2). The male structures were dried at room temperature (70-79 degrees Fahrenheit) and 20-40% humidity for 20 h. Pollen was extracted, as described in Example 2, and applied to non-induced female flowers on the same plant, C2, to produce selfed seed. Final seed counts are reported in Table 2.

### Example 4

### Production of Cannabis sativa Seed from Untreated Female Plants Pollinated with Pollen from Chemically Induced Male Flowers

Controlled pollinations between AVG treated and untreated strains were made, as shown in Table 3, to demonstrate utility to create novel genetic combinations in a breeding program or to produce hybrid seed. Two individuals from two different untreated strains, Strains F and G, were used as female recipients for crosses from various pollen collections off of strains with chemically induced male flowers. Individual female inflorescences from an untreated female plant from Strain G, plant G2, received controlled pollinations from 5 distinct pollen collections from Strain D, plant D1, as shown in Table 3. Plant G2 was also the recipient of two controlled crosses from Strain C, plant C2, Table 3. Strain G, plant G3, received controlled pollinations on female inflorescences from three distinct pollen collections from Strain D, plant D1, as shown in Table 3. Plant G3 also received a controlled pollination from Strain E, plant E2, which produced a small amount of pollen. Seed was harvested from each cross and the seed return is summarized in Table 3.

TABLE 3
Summary of crosses and resulting seed counts from untreated female plants pollinated
with pollen from chemically induced male flowers.

| | Pollination | Female × | | Seed |
|---|---|---|---|---|
| CROSS # | Type | Induced Male | Comment | Count |
| 29 | Cross | G2 × D1 | Fresh pollen, high quality, large | 145 |
| | | | amount used on multiple inflorescences | |
| 30 | Cross | G2 × D1 | Fresh pollen, high quality, large amount | 49 |
| 31 | Cross | G2 × C2 | 20 h dried anthers, good quality pollen | 61 |
| 32 | Cross | G2 × D2 | 20 h dried anthers, good quality | 94 |
| 33 | Cross | G2 × D1 | 24 h dried anthers, good quality | 12 |
| 34 | Cross | G2 × D1 | 48 h dried anthers, pollen quality good | 4 |
| 35 | Cross | G2 × C2 | 24 h dried anthers, small amount | no data |
| | | | applied to small inflorescence- Could | |
| | | | not find at harvest | |
| 36 | Cross | G3 × D1 | Fresh pollen, high quality used on | 245 |
| | | | multiple inflorescences | |
| 37 | Cross | G3 × D1 | Fresh pollen, high quality used on | 142 |
| | | | multiple inflorescences | |
| 38 | Cross | G3 × E2 | Fresh pollen, small amount and | 7 |
| | | | moderate quality | |
| 39 | Cross | G3 × D1 | 20 h dried anthers, good quality | 20 |
| 52 | Cross | F2 × D1 | 36 h dried anthers, used on multiple | 145 |
| | | | inflorescences | |
| 53 | Cross | F2 × D1 | 24 h dried anthers, small amount | 9 |
| | | | used on one small inflorescence | |
| 54 | Cross | F2 × C1 | 48 h dried anthers, low quality | 1 |
| | | | pollen | |
| 55 | Cross | F2 × M1 | 24 h dried anthers, low quality pollen | 1 |
| | | | and very little | |
| 56 | Cross | F2 × D1 | 24 h dried anthers, small amount | 1 |
| | | | applied to one inflorescence | |
| 57 | Cross | F2 × D2 | 24 h dried anthers, good quality | 28 |
| | | | pollen | |
| 58 | Cross | F3 × D1 | Fresh pollen, high quality | 79 |
| 59 | Cross | F3 × D1 | 36 h dried anthers, large volume | 147 |
| | | | applied to multiple inflorescences | |
| 60 | Cross | F3 × D1 | 24 h dried anthers, good quality | 41 |
| 61 | Cross | F3 × D1 | 24 h dried anthers, moderate quality | 7 |
| | | | pollen | |

Individual female inflorescences from an untreated female plant from Strain F, plant F2, received controlled pollinations from 3 distinct pollen collections from Strain D, plant D1, as shown in Table 3. Plant F2 also received pollen from Strain D, plant D2, Strain C, plant C2 and Strain E, plant E1, as shown in Table 3. Plant F3 received controlled pollinations from four distinct pollen collections from Strain D, Plant D1, as shown in Table 3.

### Example 5

### Production of Cannabis sativa Seed from Controlled Crosses Using Pollen from Chemically Induced Flowers and Applied to Non-Converted Female Flowers on the Sample Treated Plant

To demonstrate that the creation of novel genetic combinations for a breeding program or hybrid seed can be produced between two chemically induced strains, numerous crosses were performed. Controlled pollinations between AVG treated male donors and inflorescences that remained female on AVG treated plants were made, as shown in Table 4. Additionally, the crosses were made over time to determine the duration of viability of pollen produced from chemically induced male flowers and the receptivity of non-induced female flowers on plants treated with AVG chemistry. Pollen was collected and applied, as described in Example 2. Seed was harvested from each cross and the seed return is summarized in Table 4.

TABLE 4
Summary of crosses and resulting seed counts from using pollen from chemically
induced flowers and applied to non-converted female flowers on the sample treated plant.

| CROSS | Pollination | Female × | | Seed |
|---|---|---|---|---|
| # | Type | Induced Male | Comment | Count |
| 1 | Cross | B1 × A2 | Fresh pollen, good quality | 44 |
| 2 | Cross | B1 × A2 | 36 h dried anthers, small amount | 30 |
| 3 | Cross | B1 × C2 | 36 h dried anthers, good amount of | 35 |
| | | | pollen | |
| 4 | Cross | B1 × D1 | Fresh pollen, good quality and | 75 |
| | | | quantity | |
| 5 | Cross | B1 × A2 | Fresh pollen, low quantity | 49 |
| 6 | Cross | B1 × A2 | Fresh pollen, high quality pollen | 67 |
| 8 | Cross | B1 × C2 | Fresh pollen, high quality pollen, | 339 |
| | | | many inflorescences pollinated | |
| 9 | Cross | B1 × D1 | Fresh, high quality pollen | 51 |
| 10 | Cross | B2 × C2 | Fresh, high quality pollen | 79 |
| 11 | Cross | B2 × A2 | Fresh, low quantity pollen | 26 |
| 12 | Cross | B2 × D1 | 36 h dried anthers, low quality | 19 |
| | | | pollen | |
| 13 | Cross | B2 × D2 | Fresh, high quality pollen | 67 |
| 14 | Cross | E2 × A2 | Fresh pollen, moderate quality, | 16 |
| | | | small inflorescence pollinated | |
| 15 | Cross | E2 × A2 | 48 h dried anthers, low quality | 32 |
| | | | pollen | |
| 16 | Cross | E2 × A2 | 48 h dried anthers, very low | 3 |
| | | | volume pollen | |
| 17 | Cross | E2 × C2 | 36 h dried anthers, poor quality | 4 |
| | | | pollen | |
| 18 | Cross | E2 × C2 | 24 h dried anthers, moderate | 17 |
| | | | quality pollen | |
| 19 | Cross | E2 × D1 | Fresh, high quality pollen | 49 |
| 20 | Cross | E2 × D2 | 20 h dried anthers, high quality | 57 |
| | | | pollen | |
| 22 | Cross | E1 × C2 | Fresh pollen, moderate quality | 13 |
| | | | pollen | |
| 23 | Cross | E1 × C2 | 48 h dried anthers, low quality | 8 |
| | | | pollen | |
| 24 | Cross | E1 × D1 | Fresh, high quality pollen | 42 |
| 25 | Cross | E1 × C1 | Fresh, high quality pollen | 9 |
| 26 | Cross | E1 × C2 | 24 h dried anthers, moderate | 1 |
| | | | quality pollen used on multiple | |
| | | | inflorescences | |
| 27 | Cross | El × D1 | 24 h dried anthers, large volume of | 20 |
| | | | pollen | |
| 28 | Cross | El × D1 | Fresh, high quality pollen | 47 |

### Example 6

### Large Scale Production of Cannabis sativa Hybrid Seed Using an Ultrasonic Dehiscence System

To demonstrate the ability to produce hybrid seed efficiently, an ultrasonic dehiscence system was developed to release the pollen from chemically induced males in a cloud, while in proximity of the recipient plants. Plants D1 and D2 of the highly responsive Strain D were placed in proximity of plants B1 and B2 of Strain B that remained female, as they were unresponsive to the AVG chemistry. Ultrasonic devices (˜100-500 Hz) were used to mobilize clouds of pollen that drifted onto the female Strain B plants to pollinate them. The device was applied to the main stem, branch stems, and/or inflorescences to induce pollen release. Seed was harvested from each cross and the seed return is summarized in Table 5.

TABLE 5
Summary of crosses and resulting seed counts from crosses using
an ultrasonic vibration system.

| CROSS | Pollination | Female × | | Seed |
|---|---|---|---|---|
| # | Type | Induced Male | Comment | Count |
| 7 | Ultrasonic | B1 × C2 | Pollen visibly streaming off plant | 225 |
| | vibe cross | | with ultrasonic vibration | |
| 63 | Ultrasonic | B2 × D1 | Pollen visibly streaming off plant | 348 |
| | vibe cross | | with ultrasonic vibration | |

### Example 7

### Large Scale Production of Self Seed Using an Ultrasonic Dehiscence System on Cannabis sativa Plants

To demonstrate the ability to obtain self seed efficiently over an entire plant, an ultrasonic dehiscence system was developed to easily release pollen from the chemically induced males in a cloud around the plant. An ultrasonic device (˜100-500 Hz) was used to vibrate out clouds of pollen from plant A1 from Strain A, by touching the device to the stem, branches and induced male inflorescences. The plant was isolated from other plants to ensure purity. Some female inflorescences on plant A1 were used for controlled crosses of A2 pollen onto A1, and these inflorescences were protected and excluded from the seed count for selfing through the ultrasonic dehiscence system. Seed was harvested and the seed return is summarized in Table 6.

TABLE 6
Summary of crosses and resulting seed counts from self crosses using an ultrasonic
vibration system.

| | Pollination | | | |
|---|---|---|---|---|
| CROSS # | Type | Female × Induced Male | Comment | Seed Count |
| 64 | Ultrasonic | D2 × D2 | Pollen visibly streaming off plant | 253 |
| | vibe self | | with ultrasonic vibration | |
| 65 | Ultrasonic | C2 × C2 | Pollen visibly streaming off plant | 139 |
| | vibe self | | with ultrasonic vibration | |
| 66 | Ultrasonic | C1 × C1 | Pollen visibly streaming off plant | 27 |
| | vibe self | | with ultrasonic vibration | |
| 67 | Ultrasonic | A2 × A2 | Pollen visibly streaming off plant | 41 |
| | vibe self | | with ultrasonic vibration | |
| 68 | Ultrasonic | D1 × D1 | Pollen visibly streaming off plant | 171 |
| | vibe self | | with ultrasonic vibration | |
| 69 | Ultrasonic | A1 × A1 | Pollen visibly streaming off plant | 41 |
| | vibe self | | with ultrasonic vibration | |

### Example 8

### Validation of the Production of Feminized Cannabis sativa Seed

To demonstrate that the seed produced from the pollinations described in examples 3-8 was feminized, meaning only female plants were derived from crosses, a subset of seeds were planted from some crosses and the resulting plants were scored for gender. Plants were grown under a vegetative photoperiod (18 h day, 6 h night) until the first true leaves were formed, at which time a sample cotyledon was taken and sent for molecular sex testing to verify the progeny were exclusively female. Results are summarized in Table 7. Alternatively, plants are grown under a vegetative photoperiod (18 h day, 6 h night) until the 7th palmate leaf is observed and are then shifted to a reproductive photoperiod (12 h day and 12 h night) to induce flowering. Plants are scored for gender 7 days after the switch to a floral inductive photoperiod. Similarly, seedlings derived from the remaining crosses are scored for sex.

TABLE 7
Molecular sex test results for progeny.

| Cross | Cross | | |
|---|---|---|---|
| # | Description | Progeny ID | Sex (F/M) |
| 1 | B1 × A2 | 1.1 | F |
| | | 1.2 | F |
| | | 1.3 | F |
| | | 1.4 | F |
| | | 1.5 | F |
| 2 | B1 × A2 | 2.1 | F |
| | | 2.2 | F |
| | | 2.3 | F |
| 3 | B1 × C2 | 3.1 | F |
| | | 3.2 | F |
| | | 3.3 | F |
| 4 | B1 × D1 | 4.1 | F |
| 5 | B1 × A2 | 5.1 | F |
| | | 5.2 | F |
| | | 5.3 | F |
| | | 5.4 | F |
| | | 5.5 | F |
| 6 | B1 × A2 | 6.1 | F |
| | | 6.2 | F |
| | | 6.3 | F |
| | | 6.4 | F |
| | | 6.5 | F |
| 7 | B1 × C2 | 7.1 | F |

### Example 9

### Treatment of Genetically Female Cannabis sativa Plants with AVG as a Nodal Spray, Apical Drip or Root Drench to Induce Male Flowers

To compare delivery methods of AVG onto genetically female Cannabis sativa plants for induction of male flowers, three different modes of delivery were tested on two different strains, L and M. Plants were grown in 4 L pots outdoors under long days (˜15.5 h) until they were approximately 90 cm tall and were then transferred to a floral inductive photoperiod (12 h day/12 h night). Soon after the onset of flowering the plants were treated with 1) 200 ml root drench to the base of the plant, 2) 50 ml spray (AVG dissolved in 0.01% Tween-80) focused on the nodes with developing inflorescences or 3) 2 ml of AVG solution (AVG dissolved in 0.01% Tween-80) dripped solely on the apex of the plant with gentle massage for coverage of all tissues. For each strain and treatment, one plant was treated only one time and the other was treated 3 times, with 6 to 7 days between treatments. For the nodal spray treatment, one branch was covered during the spray application (untreated control, UTC) to test if direct contact with the solution was necessary for conversion, or if the chemistry was translocated from treated tissues to untreated tissues. Treatment descriptions are summarized in Table 8. Male structures developed, but the plants matured before pollen could form.

TABLE 8
Treatments in comparison of AVG delivery methods.

| Strain | Plant | Treatment | Number of | AVG | |
|---|---|---|---|---|---|
| Code | ID | Type | Treatments | mg/vol/Plant | |
| L | Ll | Drench | 1 | 12 | mg/200 ml |
| | L2 | Drench | 3 | 12 | mg/200 ml |
| | L3 | Spray | 1 | 12 | mg/50 ml |
| | L4 | Spray | 3 | 12 | mg/50 ml |
| | L5 | Apex Drip | 1 | 0.5 | g/2 ml |
| | L6 | Apex Drip | 3 | 0.5 | g/2 ml |
| M | M1 | Drench | 1 | 12 | mg/200 ml |
| | M2 | Drench | 3 | 12 | mg/200 ml |
| | M3 | Spray | 1 | 12 | mg/50 ml |
| | M4 | Spray | 3 | 12 | mg/50 ml |
| | M5 | Apex Drip | 1 | 0.5 | g/2 ml |
| | M6 | Apex Drip | 3 | 0.5 | g/2 ml |

### Example 10

### Effects of Different Delivery Modes of AVG on Cannabis sativa Plants

When AVG was applied as a spray or a drip directly to apical or axillary nodes, chemically induced male flowers were only observed on treated tissues, except in one instance which was due to a known contamination, demonstrating that AVG is not mobile when applied to the surface of the plant. Interestingly, when AVG is applied as a root drench, it is easily mobilized to rapidly growing sink tissues, as evidenced by localized chlorosis and the formation of chemically induced male flowers at all nodes. The root drench delivery of AVG was most efficient and had the most penetrant response. To quantitatively assess the response to the three different delivery methods of AVG, mature plants we cut down and every node on a given branch was scored for having 1) only female flowers, 2) both male and female flowers or 3) exclusively male flowers and is summarized in Tables 9 to 20.

TABLE 9
Scoring of flower sex at each node of Strain L treated with AVG
on the apical node a single time.
Strain L: Single Apical Treatment

| | Nodes | Nodes with | Nodes with | |
|---|---|---|---|---|
| Branch | with Male | Male & Female | Female | Vegetative |
| # | Flowers | Flowers | Flowers | Nodes |
| 1 | 0 | 0 | 25 | 0 |
| 2 | 0 | 0 | 28 | 0 |
| 3 | 0 | 0 | 25 | 0 |
| 4 | 0 | 0 | 24 | 0 |
| 5 | 0 | 0 | 27 | 0 |
| 6 | 0 | 0 | 24 | 0 |
| 7 | 0 | 0 | 25 | 0 |
| 8 | 0 | 0 | 27 | 0 |
| 9 | 0 | 0 | 22 | 0 |
| 10 | 0 | 0 | 20 | 0 |
| 11 | 0 | 0 | 16 | 0 |
| 12 | 0 | 0 | 15 | 0 |
| 13 | 0 | 0 | 14 | 0 |
| Apical | 0 | 5 | 22 | 0 |

TABLE 10
Scoring of flower sex at each node of Strain M treated with AVG
on the apical node a single time.
Strain M: Single Apical Treatment

| | Nodes | Nodes with | Nodes with | |
|---|---|---|---|---|
| Branch | with Male | Male & Female | Female | Vegetative |
| # | Flowers | Flowers | Flowers | Nodes |
| 1 | 0 | 0 | 40 | 0 |
| 2 | 0 | 0 | 39 | 0 |
| 3 | 0 | 0 | 25 | 0 |
| 4 | 0 | 0 | 21 | 0 |
| 5 | 0 | 0 | 23 | 0 |
| 6 | 0 | 0 | 17 | 0 |
| 7 | 0 | 0 | 18 | 0 |
| 8 | 0 | 0 | 17 | 0 |
| 9 | 0 | 0 | 14 | 0 |
| 10 | 0 | 0 | 9 | 0 |
| 11 | 0 | 0 | 9 | 0 |
| 12 | 0 | 0 | 5 | 0 |
| 13 | 0 | 0 | 5 | 0 |
| Apical | 0 | 12 | 4 | 0 |

TABLE 11
Scoring of flower sex at each node of Strain L treated with AVG
on the apical node three times.
Strain L: Three Apical Treatments

| | Nodes | Nodes with | Nodes with | |
|---|---|---|---|---|
| Branch | with Male | Male & Female | Female | Vegetative |
| # | Flowers | Flowers | Flowers | Nodes |
| 1 | 0 | 0 | 18 | 0 |
| 2 | 0 | 0 | 22 | 0 |
| 3 | 0 | 0 | 13 | 0 |
| 4 | 0 | 0 | 25 | 0 |
| 5 | 0 | 0 | 24 | 0 |
| 6 | 0 | 0 | 21 | 0 |
| 7 | 0 | 0 | 26 | 0 |
| 8 | 0 | 0 | 20 | 0 |
| 9 | 0 | 0 | 16 | 0 |
| 10 | 0 | 0 | 17 | 0 |
| 11 | 0 | 0 | 14 | 0 |
| 12 | 0 | 0 | 14 | 0 |
| 13 | 0 | 0 | 13 | 0 |
| Apical | 0 | 21 | 2 | 0 |

TABLE 12
Scoring of flower sex at each node of Strain M treated with AVG
on the apical node three times.
Strain M: Three Apical Treatments

| | Nodes | Nodes with | Nodes with | |
|---|---|---|---|---|
| Branch | with Male | Male & Female | Female | Vegetative |
| # | Flowers | Flowers | Flowers | Nodes |
| 1 | 3 | 0 | 23 | 0 |
| 2 | 0 | 0 | 18 | 0 |
| 3 | 0 | 0 | 21 | 0 |
| 4 | 0 | 0 | 17 | 0 |
| 5 | 0 | 0 | 23 | 0 |
| 6 | 0 | 0 | 22 | 0 |
| 7 | 0 | 0 | 17 | 0 |
| 8 | 0 | 0 | 7 | 0 |
| 9 | 0 | 0 | 6 | 0 |
| 10 | 0 | 0 | 9 | 0 |
| 11 | 0 | 0 | 11 | 0 |
| 12 | 0 | 0 | 7 | 0 |
| Apical | 0 | 11 | 3 | 0 |

TABLE 13
Scoring of flower sex at each node of Strain L treated
with AVG as a single spray.
Strain L: Single Plant Spray

| | Nodes | Nodes with | Nodes with | |
|---|---|---|---|---|
| Branch | with Male | Male & Female | Female | Vegetative |
| # | Flowers | Flowers | Flowers | Nodes |
| 1 | 0 | 33 | 3 | 0 |
| 2 | 0 | 9 | 27 | 0 |
| 3 | 0 | 15 | 11 | 0 |
| 4 | 0 | 16 | 14 | 0 |
| 5 | 0 | 8 | 16 | 0 |
| 6 | 0 | 14 | 12 | 0 |
| 7 | 0 | 11 | 11 | 0 |
| 8 UTC | 0 | 3 | 20 | 0 |
| 9 | 0 | 6 | 9 | 0 |
| 10 | 0 | 6 | 8 | 0 |
| 11 | 0 | 6 | 5 | 0 |
| 12 | 0 | 3 | 6 | 0 |
| Apical | 0 | 12 | 4 | 0 |

TABLE 14
Scoring of flower sex at each node of Strain M treated with AVG as a
single spray.
Strain M: Single Plant Spray

| | Nodes with | Nodes with | Nodes with | |
|---|---|---|---|---|
| Branch | Male | Male & Female | Female | Vegetative |
| # | Flowers | Flowers | Flowers | Nodes |
| 1 | 0 | 21 | 4 | 0 |
| 2 | 0 | 24 | 8 | 0 |
| 3 | 0 | 19 | 8 | 0 |
| 4 | 0 | 19 | 10 | 0 |
| 5 | 0 | 5 | 8 | 0 |
| 6 | 0 | 7 | 10 | 0 |
| 7 | 0 | 0 | 17 | 0 |
| UTC | | | | |
| 8 | 0 | 8 | 7 | 0 |
| 9 | 0 | 5 | 7 | 0 |
| 10 | 0 | 3 | 7 | 0 |
| 11 | 0 | 4 | 8 | 0 |
| 12 | 0 | 6 | 0 | 0 |
| Apical | 0 | 12 | 6 | 0 |

TABLE 15
Scoring of flower sex at each node of Strain L treated with AVG as a
spray three times.
Strain L: Three Plant Sprays

| | Nodes with | Nodes with | Nodes with | |
|---|---|---|---|---|
| Branch | Male | Male & Female | Female | Vegetative |
| # | Flowers | Flowers | Flowers | Nodes |
| 1 | 0 | 37 | 10 | 0 |
| 2 | 0 | 15 | 4 | 0 |
| 3 | 0 | 11 | 15 | 0 |
| 4 | 0 | 25 | 8 | 0 |
| 5 | 0 | 26 | 2 | 0 |
| 6 | 0 | 21 | 7 | 0 |
| 7 | 0 | 22 | 6 | 0 |
| 8 | 0 | 0 | 22 | 0 |
| UTC | | | | |
| 9 | 0 | 17 | 4 | 0 |
| 10 | 0 | 21 | 4 | 0 |
| 11 | 0 | 14 | 0 | 0 |
| 12 | 0 | 22 | 0 | 0 |
| 13 | 0 | 13 | 1 | 0 |
| 14 | 0 | 11 | 0 | 0 |
| Apical | 0 | 23 | 0 | 0 |

TABLE 16
Scoring of flower sex at each node of Strain M treated with AVG as a
spray three times.
Strain M: Three Plant Sprays

| | Nodes with | Nodes with | Nodes with | |
|---|---|---|---|---|
| Branch | Male | Male & Female | Female | Vegetative |
| # | Flowers | Flowers | Flowers | Nodes |
| 1 | 0 | 52 | 0 | 0 |
| 2 | 0 | 23 | 3 | 0 |
| 3 | 0 | 28 | 0 | 0 |
| 4 | 0 | 16 | 3 | 0 |
| 5 | 0 | 11 | 5 | 0 |
| 6 | 0 | 17 | 0 | 0 |
| 7 | 0 | 0 | 12 | 0 |
| UTC | | | | |
| 8 | 0 | 13 | 0 | 0 |
| 9 | 0 | 10 | 0 | 0 |
| 10 | 0 | 6 | 4 | 0 |
| 11 | 0 | 6 | 0 | 0 |
| Apical | 0 | 14 | 0 | 0 |

TABLE 17
Scoring of flower sex at each node of Strain L treated with AVG as a
single root drench.
Strain L: Single Root Drench

| | Nodes with | Nodes with | Nodes with | |
|---|---|---|---|---|
| Branch | Male | Male & Female | Female | Vegetative |
| # | Flowers | Flowers | Flowers | Nodes |
| 1 | 0 | 18 | 7 | 0 |
| 2 | 6 | 18 | 6 | 0 |
| 3 | 11 | 8 | 29 | 0 |
| 4 | 0 | 14 | 8 | 0 |
| 5 | 0 | 16 | 7 | 0 |
| 6 | 0 | 30 | 1 | 0 |
| 7 | 0 | 13 | 20 | 0 |
| 8 | 0 | 6 | 20 | 0 |
| 9 | 0 | 1 | 19 | 0 |
| 10 | 0 | 2 | 17 | 0 |
| 11 | 0 | 4 | 14 | 0 |
| 12 | 0 | 8 | 10 | 0 |
| 13 | 0 | 2 | 14 | 0 |
| Apical | 0 | 3 | 17 | 0 |

TABLE 18
Scoring of flower sex at each node of Strain M treated with AVG as a
single root drench.
Strain M: Single Root Drench

| | Nodes with | Nodes with | Nodes with | |
|---|---|---|---|---|
| Branch | Male | Male & Female | Female | Vegetative |
| # | Flowers | Flowers | Flowers | Nodes |
| 1 | 2 | 25 | 5 | 0 |
| 2 | 0 | 33 | 0 | 0 |
| 3 | 0 | 31 | 4 | 0 |
| 4 | 0 | 17 | 4 | 0 |
| 5 | 0 | 21 | 2 | 0 |
| 6 | 0 | 20 | 6 | 0 |
| 7 | 0 | 19 | 4 | 0 |
| 8 | 0 | 19 | 2 | 0 |
| 9 | 0 | 15 | 1 | 0 |
| 10 | 0 | 13 | 2 | 0 |
| 11 | 0 | 12 | 2 | 0 |
| 12 | 0 | 18 | 2 | 0 |
| 13 | 0 | 6 | 1 | 0 |
| Apical | 0 | 23 | 0 | 0 |

TABLE 19
Scoring of flower sex at each node of Strain M treated with AVG with
three root drenches.
Strain L: Three Root Drenches

| | Nodes with | Nodes with | Nodes with | |
|---|---|---|---|---|
| Branch | Male | Male & Female | Female | Vegetative |
| # | Flowers | Flowers | Flowers | Nodes |
| 1 | 14 | 54 | 0 | 0 |
| 2 | 9 | 24 | 0 | 0 |
| 3 | 2 | 33 | 0 | 0 |
| 4 | 6 | 30 | 3 | 0 |
| 5 | 0 | 38 | 2 | 0 |
| 6 | 3 | 23 | 7 | 0 |
| 7 | 0 | 23 | 8 | 0 |
| 8 | 0 | 24 | 12 | 0 |
| 9 | 0 | 23 | 10 | 0 |
| 10 | 0 | 10 | 14 | 0 |
| 11 | 0 | 10 | 13 | 0 |
| 12 | 0 | 8 | 8 | 0 |
| 13 | 0 | 10 | 8 | 0 |
| Apical | 0 | 32 | 5 | 0 |

TABLE 20
Scoring of flower sex at each node of Strain M treated with AVG with
three root drenches.
Strain M: Three Root Drenches

| | Nodes with | Nodes with | Nodes with | |
|---|---|---|---|---|
| Branch | Male | Male & Female | Female | Vegetative |
| # | Flowers | Flowers | Flowers | Nodes |
| 1 | 0 | 35 | 0 | 0 |
| 2 | 0 | 34 | 0 | 0 |
| 3 | 0 | 34 | 3 | 0 |
| 4 | 0 | 32 | 4 | 0 |
| 5 | 0 | 34 | 0 | 0 |
| 6 | 0 | 26 | 0 | 0 |
| 7 | 0 | 26 | 0 | 0 |
| 8 | 0 | 23 | 0 | 0 |
| 9 | 0 | 18 | 0 | 0 |
| 10 | 0 | 13 | 0 | 0 |
| 11 | 0 | 12 | 0 | 0 |
| 12 | 0 | 15 | 0 | 0 |
| 13 | 0 | 14 | 0 | 0 |
| 14 | 0 | 12 | 0 | 0 |
| 15 | 0 | 10 | 0 | 0 |
| Apical | 0 | 29 | 0 | 0 |

### Example 11

### Treatment of Cannabis sativa Plants with AVG at Two Rates

Two replicate clones of three separate strains/genotypes, H, I and J, were selected to test the effects of two different rates of multiple treatments of AVG initiated prior to the transition to flowering. Clones were transplanted into 2.2 L pots with peat-based substrate. The plants were grown under long day conditions (19 h day and 5 h night) for 35 days, when they received their first AVG treatment. The stems of the plants ranged from 6-7 mm at the time of treatment. As stem diameter is an indicator of plant size and vigor, a reduced amount of chemistry was applied compared to the experiment described in Example 1. For each strain, one replicate clone received 4.5 mg of AVG and the other received 9 mg AVG. AVG was applied as a root drench by pouring 200 ml of AVG solution near the base of the plant. Five days after the first AVG treatment, the plants were transferred to a controlled environment with a 13 h day and an 11 h night, to induce flowering. Within a week after the transition to short days (13 h), the initiation of flowering was observed. Seven and 14 days after the first treatment, the same AVG treatment, as above, was applied, for a total of three treatments.

The day after the first AVG treatment, the apical and axillary nodes showed signs of chlorosis, indicating the AVG chemistry was successfully translocated through the roots to apical parts of the plants. The amount of chlorosis observed was dose dependent, with plants treated with 9 mg of AVG showing more chlorosis than those treated with 4.5 mg of AVG. The amount of chlorosis was also strain dependent. The 4.5 mg treatment produced the strongest chlorosis in Strain J, a moderate chlorosis in strain I, and Strain H had almost no response. However, Strain H had a strong chlorosis response with the 9 mg treatment, similar to Strain I. Strain J had the most chlorosis. The chlorosis response was apparent after the first treatment only, except for strain J that had chlorosis with additional 9 mg treatments.

TABLE 21
Treatment of three Cannabis sativa strains with two rates of AVG and the
male conversion rating.

| Strain | | | Male Conversion |
|---|---|---|---|
| Code | Plant ID | mg AVG/vol/plant | Rating |
| H | H1 | 4.5 mg/200 ml | Moderate |
| | H2 | 9 mg/200 ml | Complete |
| I | I1 | 4.5 mg/200 ml | Mild |
| | I2 | 9 mg/200 ml | Moderate |
| J | J1 | 4.5 mg/200 ml | Mild |
| | J2 | 9 mg/200 ml | Moderate |

### Example 12

### Production of Male Structures, Pollen Collection and Seed Harvest on Cannabis sativa Plants

Induction of male flowers was dose dependent, with more male flowers induced on plants that received 9 mg AVG than those that received 4.5 mg AVG. Strain H was most responsive overall, and with the 9 mg treatment there was almost complete conversion to maleness. Strain I had moderate chemical induction of male flowers and strain J had mild chemical induction of male flowers. 4.5 mg of AVG induced male flowers minimally for strain J, mildly for strain I and moderately for strain H. To obtain pollen for controlled pollinations, extruded anthers or whole male flowers were collected using fine forceps. These tissues were then 1) immediately gently sieved with a 355 micron sieve or 2) allowed to dry for approximately 24 h, 36 h, or 48 h, and then sieved to release pollen. Pollen quality was assessed under a microscope (10× magnification) and clear, round pollen was scored as high quality. Clear, angular pollen was scored as moderate quality. Angular and cloudy pollen was scored as low quality. Pollen was applied to female flowers using a small paintbrush.

In addition to controlled pollinations, individual plants treated with 9 mg of AVG which had a stronger male response, were subjected to the ultrasonic dehiscence method in isolation to generate self seed. Strain H, plant 2 (H2) and strain I, plant 2 (I2) were treated with the ultrasonic dehiscence method.

Approximately 30-45 days after pollination, individual inflorescences, or stems containing multiple inflorescences, as appropriate, are removed from the plant and placed in a fine mesh bag and allowed to fully dry out for at least 7 days. After this desiccation step, the seeds are removed from the dried inflorescences through manual manipulation. Seeds are then counted and placed in tubes for storage.

Alternatively, approximately 30-45 days after pollination, the plants are allowed to dry out fully in their pots for about 3 days. Inflorescences or branches with marked controlled crosses are collected individually, allowed to dry at approximately 75-80 degrees Fahrenheit and 20-40% humidity for at least 7 days, then seeds are harvested, counted and stored. Remaining plants or branches are removed and dried similarly, and seed produced through ultrasonic vibration pollinations are harvested, counted and stored.

### Example 13

### Production of Cannabis sativa Selfed Seed Through Controlled Pollinations

Because female flowers and chemically induced male flowers were present on responsive strains, it is possible to create feminized selfed seed. To obtain a small quantity of selfed seed, pollen was collected from chemically induced male structures (yellow stamen and whole male flowers) of Strain I, plant rep number 1 (I1). This pollen was used to pollinate female inflorescences on the same plant, I1, as described in Example 2. Pollen was similarly collected from Strain I, plant rep number 2 (I2). The pollen was applied to female inflorescences on the genetically identical clone, plant I1, demonstrating an alternative method to obtain the equivalent of selfed seed. Seed was collected, counted and stored.

TABLE 22

| | | Female × | | |
|---|---|---|---|---|
| CROSS | Pollination | Induced | | Seed |
| # | Type | Male | Comment | Count |
| 12 | sib cross | I1 × I2 | 24 h dried anthers, very | 0 |
| | | | little pollen, moderate quality | |
| | | | on 1 small inflorescence | |
| 14 | self | I1 × I1 | 24 h dried anthers from a few | 0 |
| | | | flowers, small amount of | |
| | | | pollen applied to small | |
| | | | branch | |

### Example 14

### Production of Cannabis sativa Seed from Untreated Female Plants Pollinated with Pollen from Chemically Induced Male Flowers

To further demonstrate that viable male pollen can be produced on genetically female plants that are chemically induced to produce male flowers, pollen was collected as described in Example 2. Controlled pollinations were made between AVG treated male donors from strains H, I and J onto naturally female flowers of an untreated plant of strain K, (K1) as shown in Table 23. Seed was collected, counted, and stored.

TABLE 23

| | | Female × | | |
|---|---|---|---|---|
| CROSS | Pollination | Induced | | Seed |
| # | Type | Male | Comment | Count |
| 1 | cross | K1 × J2 | 18 h dried anthers, small amount | 0 |
| | | | of low quality pollen | |
| 2 | cross | K1 × H2 | 18 h dried anthers, small amount | 0 |
| | | | of low quality pollen | |
| 3 | cross | K1 × I2 | 18 h dried anthers, small amount | 0 |
| | | | of low quality pollen | |
| 4 | cross | K1 × H2 | 42 h dried anthers, a lot of | 0 |
| | | | moderate quality pollen | |
| 7 | cross | K1 × I2 | 42 h dried anthers, small amount | 0 |
| | | | of low quality pollen | |
| 10 | cross | K1 × H2 | 24 h dried anthers, large amount | 0 |
| | | | of good quality pollen put on | |
| | | | apex | |
| 13 | cross | K1 × I1 | 24 h dried anthers from a few | 0 |
| | | | flowers, small amount of pollen | |
| | | | applied to small inflorescence | |

### Example 15

### Production of Cannabis sativa Seed from Controlled Crosses (Pollen on Female Flowers of Induced Plants)

To demonstrate that the creation of novel genetic combinations for a breeding program or hybrid seed can be produced between two chemically induced strains, several crosses were performed as described in Table 24. Controlled pollinations between AVG treated male donors and inflorescences that remained female on AVG treated plants were performed. Seed was harvested as described in Example 2.

TABLE 24

| | | Female × | | |
|---|---|---|---|---|
| CROSS | Pollination | Induced | | Seed |
| # | Type | Male | Comment | Count |
| 15 | cross | J1 × I1 | 24 h dried anthers from a few | 32 |
| | | | flowers, small amount of pollen | |
| | | | applied to small branch | |
| 5 | cross | I1 × H2 | 42 h dried anthers, a lot of | 0 |
| | | | moderate quality pollen | |
| 6 | cross | J1 × H2 | 42 h dried anthers, a lot of | 0 |
| | | | moderate quality pollen | |
| 8 | cross | J1 × I2 | 42 h dried anthers, small | 0 |
| | | | amount of low quality pollen | |
| 9 | cross | I1 × H2 | 24 h dried anthers, large | 0 |
| | | | amount of good quality pollen | |
| | | | put on 2 branches | |
| 11 | cross | J1 × H2 | 24 h dried anthers, large amount | 0 |
| | | | of good quality pollen put on 2 | |
| | | | branches | |
| 16 | cross | I1 × H2 | 24 h dried anthers, large volume | 1 |
| | | | of high quality pollen applied to | |
| | | | all available flowers. Previous | |
| | | | crosses covered to avoid | |
| | | | contamination. | |
| 17 | cross | J1 × H2 | 24 h dried anthers, large volume | 0 |
| | | | of high quality pollen applied to | |
| | | | all available flowers. Previous | |
| | | | crosses covered to avoid | |
| | | | contamination. | |

### Example 16

### Induction of Male Flowers on Larger Genetically Female Cannabis sativa Plants

To demonstrate that seed could be produced using pollen generated from induced male flowers on a larger genetically female plant, plants are grown under vegetative conditions until the plants are greater than 85 cm tall, with stem diameters greater than 12 mm. Plants are grown in 8 liter pots. Pollen produced from the induced male plants is used to pollinate untreated female plants or flowers that remain female on AVG treated plants, as described in Examples 2-5. Larger plants require more active ingredient and in a larger volume to contact roots, so the dose of AVG provided is more than 15 mg active ingredient dissolved in at least 300 ml of water. Strains D, M and N are grown in a long day, vegetative photoperiod (18 h day, 6 h night), until they are greater than 85 cm tall. The first treatment of AVG is delivered by root drench about 5 days prior to transition to a short day, floral inductive photoperiod (13 h day, 11 h night). Additional AVG treatments are applied after the transition to a floral inductive photoperiod. Chemically induced male flowers and anthers are collected as described in Example 2. Pollen is collected and applied to untreated female plant or female flowers on chemically treated plants that do not completely convert to maleness. Alternatively, the ultrasonic dehiscence method is used to mobilize pollen from chemically induced male flowers to female flowers for pollination. Resulting seed are collected, counted and stored.

### Example 17

### Foliar Application of AVG to Induce Male Flowers and Viable Pollen on Cannabis sativa Plants

Genetically female plants derived from seed produced in previous examples were used to test the ability of AVG to induce the production of male flowers. Plants from two different genetic backgrounds (L and M) were grown vegetatively under 18 h of light per day for 79 days and then transferred to a floral inductive environment with 12 h of light per day. Apical portions of the plants were spray treated with 8 ml of 6.3 mg of AVG in 25 ml water per plant using a small atomizer on day 79 and day 85 after seeding. Flowering was first observed 88 days after seeding and viable male structures were observed 11 days later. Pollen was used for self and cross pollinations 110 days after seeding. One plant was highly converted and most flowers were male. Pollen from that plant was used to pollinate some of the remaining female structures on other treated plant and on an untreated plant of genotype N. Table 25 summarizes the results obtained in Example 17.

TABLE 25

| CROSS | Pollination | Female × | Seed |
|---|---|---|---|
| # | Type | Induced Male | Count |
| 123 | Cross | N1 × L1 | 5 |
| 124 | Cross | N1 × L1 | 7 |
| 125 | Self | M1 × M1 | 29 |
| 126 | Self | M1 × M1 | 19 |
| 127 | Cross | M1 × M1 | 25 |
| 128 | Cross | M1 × M1 | 62 |
| 129 | Cross | M1 × M1 | 31 |
| 130 | Cross | N1 × L1 | 0 |
| 131 | Cross | N1 × L1 | 0 |
| 132 | Cross | M1 × L1 | 29 |
| 133 | Ultrasonic | L1 × L1 | 161 |
| | Self | | |
| 134 | Self | M1 × M1 | 15 |
| 135 | Cross | M1 × L1 | 6 |
| 136 | Cross | M2 × L1 | discard |
| | | | plant |
| 137 | Ultrasonic | M1 × M1 | 36 |
| | Self | | |

### Example 18

### Chemically Induced Male Flowers on Genetically Female Cannabis sativa with Auto-Flower Trait

Cannabis sativa plants harboring the auto-flower trait are expected to flower under long day photoperiods and should not require a short-day reproductive signal to flower. To demonstrate the efficacy of AVG to induce male flowers on genetically female plants, two plants containing the auto-flower trait were grown under 18 h days. Treatments were performed at 32 (signs of first flower), 35 and 39 days after seeding with 8 ml of 4.2 mg AVG in 25 ml water per plant under 18-hour day conditions.

The first plant (Auto-1) was sprayed with AVG a fourth time with 10 ml of 6.3 mg AVG in 25 ml of water at 44 days after seeding and then moved to a 13 h day to increase the flowering response 48 days after seeding. Male flowers were first observed at 48 days after seedings and pollen was collected at 62 days after seeding. Pollen from Auto-1 was used for controlled pollinations on other plants.

The second plant (Auto-2) was moved 44 days after seeding to a 16 h day environment to slightly reduce the vegetative signal, as the flowering response was not strong. This plant was sprayed with AVG a fourth time with 10 ml of 6.3 mg AVG in 25 ml of water per plant at 44 days after seeding. This plant had a more vegetative phenotype and was moved into 13-hour day 83 days after seeding. Relatively few male structures were observed, and pollen was used for a controlled self-pollination and was allowed to naturally self-pollinate female flowers on the same plant. Seed results are summarized in Table 26.

Three additional auto-flower seeds were germinated to further demonstrate the ability of AVG to induce male structures on plants harboring the auto-flower trait. These plants were designated Auto-3, Auto-4, and Auto-5. Plants were germinated and initially grown under 18 h days. All plants received a foliar spray of 8 ml of 4.2 mg AVG in 25 ml water per plant at 25 and 29 days after seeding. Flowering was first observed at 33 days after seeding and a third foliar spray of 8 ml of 4.2 mg AVG in 25 ml water per plant was delivered 34 days after seeding. Auto-3 and Auto-5 plant both died due to root disease.

Auto-4 was moved to a 16 h day at 35 days after seeding and sprayed a fourth time with 10 ml of 6.3 mg AVG in 25 ml of water. Auto-4 then received one root drench treatment of 100 mL solution with 3.75 mg AVG 62 days after seeding. Auto-4 was then moved into 13-hour day 73 days after seeding and male flowers were observed 80 days after seeding. Auto-4 produced a significant number of male flowers with viable pollen that was used in a controlled cross and to self-pollinate, resulting in seed set. Seed results are summarized in Table 26.

TABLE 26

| CROSS | Pollination | Female | Induced | | Seed |
|---|---|---|---|---|---|
| # | Type | Recipient | Male | Comment | Count |
| 169 | Self | Auto-4 | — | Pollen mobilized to self | 331 |
| | | | | remaining open flowers | |
| 172 | Controlled | Auto-2 | self | Pollen applied to | 8 |
| | Self | | | marked nodes | |
| 176 | Cross | Auto-2 | Auto-1 | Pollen applied to | 8 |
| | | | | marked nodes | |
| 177 | Cross | Auto-2 | Auto-4 | Pollen applied to | 1 |
| | | | | marked nodes | |
| 178 | Cross | Auto-2 | Auto-4 | Pollen applied to | 113 |
| | | | | marked nodes | |
| 183 | Controlled | Auto-4 | — | Pollen applied to | 508 |
| | Self | | | marked nodes | |
| 185 | Self | Auto-2 | — | Pollen mobilized to self | 20 |
| | | | | remaining open flowers | |

### Example 19

### Chemically Induced Male Flowers and Seed Production with a 1-MCP Root Drench

To determine if the ethylene inhibitor 1-Methylcyclopropene (1-MCP) can induce male flowers on genetically female Cannabis ssp plants, 1-MCP was applied at two different rates to 3 different varieties, with 2 replicates per treatment. The plants were treated with 1-MCP three times, with the first treatment 51 days after seeding, which was on the day plants were transitioned from a vegetative, long day photoperiod (18 h day) to a reproductive, short day photoperiod (12 h day), the second treatment was applied 7 days later and the third treatment was 8 days after the second. For the first two treatments the low rate was 0.27 mM 1-MCP and 25 ml solution per plant was immediately applied as a root drench. The high rate was 0.54 mM and similarly, 25 ml of solution was applied as a root drench. For the third treatment, the dose was doubled by applying 50 ml of the solutions described above, as the plants were larger. While mild chlorosis of growing apices was observed, which is an expected response with the application of an ethylene inhibitor, no clear or consistent male structures were observed in any of the treatments.

### Example 20

### Chemically Induced Male Flowers and Seed Production: Treatment Number

To demonstrate that a single AVG root drench treatment delivered after a shift to a reproductive environment was sufficient to induce a female Cannabis clone to produce male flowers, a clone of variety Q was given a single treatment 1 week after being moved to a reproductive 12 hour day environment. The Q clone was grown under vegetative conditions (18 h day) until the stem was about 10 mm in diameter, at which time it was transferred to floral inductive conditions (12 h day). After 7 days of growth in a floral inductive environment, and after signs of flowering were present, the main stem diameter was 11 mm. A single drench treatment containing 19 mg of AVG in 300 ml water was delivered to the roots at this time. Approximately 95% of the flowers were converted to male flowers that produced significant pollen one month after the treatment. An untreated female plant of another variety (O) was used as a recipient for Q pollen, using the ultrasonic dehiscent system two times (one day in between pollination events). This system was highly effective at mobilizing the pollen to completely pollinate the female plant in a matter of seconds. Pollen was also manually collected an applied to 2 other recipients, P and L. These data demonstrate a single treatment of AVG can be sufficient to convert a genetically female plant almost completely to a male plant phenotypically. Seed counts are reported in Table 27.

TABLE 27

| | | | Female × | |
|---|---|---|---|---|
| CROSS | Pollination | | Induced | Seed |
| # | Type | Pollination Method | Male | Count |
| 153 | Cross | Ultrasonic Dehiscence | O1 × Q1 | 1984 |
| | | System | | |
| 154 | Cross | Limited, controlled | P1 × Q1 | 26 |
| | | pollination | | |
| 155 | Cross | Limited, controlled | L1 × Q1 | 123 |
| | | pollination | | |

### Example 21

### 2-Aminoisobutyric Acid (AIB)

To determine if the ethylene inhibitor 2-Aminoisobutyric acid (AIB) can induce male flowers on genetically female Cannabis ssp plants, three different rates of 2-Aminoisobutyric acid (AIB) were applied to plants as a root drench, with two applications per rate. The first application occurred 79 days after seeding, which was on the day plants were transitioned from a vegetative, long day photoperiod (18 h day) to a reproductive, short day photoperiod (12 h day), and the second treatment was applied 5 days later. The stem diameter of the plants on the day of the first treatment were 9 to 10 mm and the plants ranged from 37 to 54 cm in height. The three rates tested were 50, 150 and 300 mM AIB in water, and 150 ml of solution was applied to the roots as a soil drench. Mild phytotoxicity was observed in leaf and meristematic tissues within 2 days of the first treatment. After the second treatment, more severe phototoxicity was observed, particularly in the highest rate, of which the plant ultimately died. The plants treated with 50 and 150 mM AIB survived and produced small male structures that did not produce viable pollen.

### Example 22

### Chemically Induced Male Flowers and Seed Production: 2-Aminoisobutyric Acid (AIB)

To determine the effect of treatment number and rate of AVG on the ability to induce male flowers on genetically female Cannabis sativa plants. Cloned plants of genotype P were treated 1, 3 or 8 times with an AVG drench, at correspondingly high, medium or low rates. The plants were 45-54 cm tall with a stem diameter of 9 mm at the time of the first treatment day, Day 1. The plant treated 8 times (8×) received its first treatment on Day 1, and subsequent treatments occurred every other day until 8 treatments were delivered over a 15 day period. The 8× plant was switched to 13-hour day on day 7 of the treatment time course. The plant that received 3 treatments (3×) received its first treatment on Day 2, with following treatments one week apart, finishing on Day 22. The 3× plant was switched to 13-hour day on day 6 of the treatment time course. The single treatment (1×) was delivered on Day 6, which was the day before plants were switched from a vegetative photoperiod (16 h day) to a reproductive photoperiod (13 h day). Plants treated 8 times, received a low dose of 11 ppm 2.25 mg AVG in 200 ml water on each treatment, plants treated 3 times received 9 mg AVG in 300 ml water on each treatment, and the plant treated one time received 21 mg AVG in 300 ml water. On Day 25, all plants showed some signs of maleness, with the 1× and 3× plants showing a stronger response, with ultimately about 80% conversion. The 8× treated plant had about 50% conversion, with fewer viable male structures. Pollen from treated plants was used to pollinate female plants not treated with AVG of germplasm Q or P. Seed results are summarized in Table 28.

TABLE 28

| Cross | Pollination | Female | Induced | | Seed |
|---|---|---|---|---|---|
| # | Type | Recipient | Male | Comment | Count |
| 91 | Controlled | Q7 | P-3X AVG | Little pollen | 0 |
| | Cross | | | | |
| 93 | Controlled | Q7 | P-3X AVG | 2 inflorescences | 6 |
| | Cross | | | pollinated | |
| 95 | Controlled | Q7 | P-8X AVG | Young flowers | 3 |
| | Cross | | | | |
| 96 | Controlled | Q7 | P-1X AVG | 1 inflorescence | 3 |
| | Cross | | | pollinated | |
| 97 | Controlled | Q7 | P-3X AVG | 2 inflorescences | 5 |
| | Cross | | | pollinated | |
| 101 | Controlled | Q9 | P-3X AVG | Many flowers | 37 |
| | Cross | | | pollinated | |
| 102 | Controlled | Q8 | P-3X AVG | Many flowers | 9 |
| | Cross | | | pollinated | |
| 103 | Controlled | Q8 | P-1X AVG | Female flowers | 0 |
| | Cross | | | young | |
| 105 | Controlled | Q9 | P-3X AVG | 72 h pollen | 16 |
| | Cross | | | | |
| 106 | Controlled | Q9 | P-1X AVG | 4 day-old pollen | 8 |
| | Cross | | | | |
| 111 | Controlled | Q9 | P-3X AVG | Fresh pollen | 15 |
| | Cross | | | | |
| 112 | Controlled | P1 | P-3X AVG | Small inflorescences | 3 |
| | Cross | | | | |
| 113 | Controlled | P1 | P-3X AVG | Small inflorescences | 1 |
| | Cross | | | | |

The breadth and scope of the present disclosure should not be limited by any of the above-described exemplary embodiments, but should be defined only in accordance with the following claims and their equivalents.

### REFERENCES

1. Rudich, J., Zamski, E., 1985. Citrullus lanatusIn: Halevy, A. (Ed.) Handbook of Flowering, vol. 2. CRC Press, Boca Raton, Fla., pp 272-274.
2. Sugiyama, K., Kanno, T., Morishita, M., 1998. Evaluation method of female flower bearing ability in watermelon using silver thiosulfate (STS). J. Jpn. Soc. Hortic Sci. 67, 185-89.
3. Lubell, J. D. and Brand, M. H., 2018. Foliar Sprays of Silver Thiosulfate Produce Male Flowers on Female Hemp Plants. American Society for Horticulture Science 28(6), 743-747.
4. Ram, H. and Sett R. 1982. Induction of fertile male flowers in genetically female Cannabis sativa plants by silver nitrate and silver thiosulphate anionic complex. Theoretical and Applied Genetics 62, 369-375.
5. Ram, H. and Set, R. 1981. Modification of Growth and Sex Expression in Cannabis sativa by Aminoethoxyvinylglycine and Ethephon. Z. Pflanzenphysiol. Bd 105, 165-172.
6. Schaller G. E., Binder B. M. 2017. Inhibitors of Ethylene Biosynthesis and Signaling. In: Binder B., Eric Schaller G. (eds) Ethylene Signaling. Methods in Molecular Biology, vol 1573. Humana Press, New York, N.Y.
7. Tacuri, M.; Flores, D.; Anrango, M. J.; Koch, A.; Naranjo, B.; and Cumbal, L., 2016. Novel Technique for Degradation for Silver Thiosulfate Present in Wastewater of the Post-Harvest Treatment of Ethylene-Sensitive Flowers. Biology and Medicine. 8, 278-283.

What is claimed is:

1. A method of producing a genetically female Cannabis sativa seed, the method comprising:
a) inducing one or more male flowers on a genetically female Cannabis sativa plant by applying the chemical ethylene inhibitor aminoethoxyvinylglycine (AVG) to the plant, wherein the female plant forms one or more pollen-producing fertile male flowers; and
b) producing the seed by pollinating a female flower with pollen from the induced one or more male flowers,
wherein the seed produced is genetically female.

2. The method of claim 1, wherein the seed is produced by self-pollination of pollen from the induced one or more male flowers onto a female flower of the same plant.

3. The method of claim 1, wherein the seed is produced by cross-pollination of pollen from the induced one or more male flowers onto a female flower of a separate female plant.

4. The method of claim 3, wherein the separate female plant used for cross-pollination is genetically identical to the plant with the induced male flowers.

5. The method of claim 3, wherein the separate female plant used for cross-pollination has a genotype that is distinct from the plant with the induced male flowers;
optionally, wherein the separate female plant used having a genotype that is distinct from the plant with the induced male flowers is from a distinct inbred variety.

6. The method of claim 2 or 3, wherein the pollen from the induced one or more male flowers is disbursed for pollination by an ultrasonic dehiscence system.

7. The method of any one of claims 3 to 6, wherein the cross-pollination creates inbred seed.

8. The method of any one of claims 3 to 6, wherein the cross-pollination creates hybrid seed.

9. The method of any one of claims 1 to 8, wherein AVG is applied to the plant in at least two, three, or four separate applications.

10. The method of any one of claims 1 to 9, wherein AVG is applied in at least one application by root drench to the plant.

11. The method of any one of claims 1 to 10, wherein AVG is applied in at least one application by foliar spray to the plant,
optionally, wherein the foliar spray is applied to apical and axillary nodes at a rate of 0.12 mg to 0.4 mg AVG per ml of 0.01% of Tween 80 solution.

12. The method of any one of claims 9 to 11, wherein AVG is applied to the plant and:
(i) wherein the application comprises root drench in all applications;
(ii) wherein the application comprises root drench but not foliar spray in any applications;
(iii) wherein the application comprises foliar spray in all applications;
(iv) wherein the application comprises foliar spray but not root drench in any applications;
(v) wherein the application comprises root drench combined with foliar spray in all applications;
(vi) wherein the application comprises root drench combined with foliar spray in at least one application and root drench but not foliar spray in at least one separate application;
(vii) wherein the application comprises root drench combined with foliar spray in at least one application and foliar spray but not root drench in at least one separate application; and/or
(viii) wherein the application comprises root drench but not foliar spray in at least one application and also comprises foliar spray but not root drench in at least one separate application.

13. The method of claim 12, wherein in the first application of AVG to the plant comprises application by root drench;
optionally, wherein the first application of AVG to the plant comprises root drench but not foliar spray.

14. The method of claim 12, wherein the first application of AVG to the plant comprises application by foliar spray;
optionally, wherein the first application of AVG to the plant comprises foliar spray but not root drench.

15. The method of any one of claims 1 to 14, wherein AVG is applied to the plant prior to flowering;
optionally, wherein AVG is first applied to the plant prior to flowering;
optionally, wherein AVG is only applied to the plant prior to flowering.

16. The method of claim 15, wherein AVG is applied to the plant in at least two, three, or four separate applications prior to flowering.

17. The method of any one of claims 1 to 16, wherein AVG is applied to the plant after flowering is initiated,
optionally, wherein AVG is applied to the plant in at least two, three, or four, separate applications after flowering is initiated,
optionally, wherein AVG is only applied to the plant after flowering is initiated.

18. The method of any one of claims 1 to 17, wherein AVG is applied to the plant both prior to flowering and after flowering is initiated.

19. The method of claim 18, wherein:
(i) the AVG application comprises root drench prior to flowering and separately comprises root drench after flowering is initiated,
optionally, wherein the AVG application comprises at least one root drench application without foliar spray prior to flowering, further optionally, wherein the AVG application comprises root drench but not foliar spray prior to flowering, and/or
optionally, wherein the AVG application separately comprises at least one root drench application without foliar spray after flowering is initiated, further optionally, wherein the AVG application comprises root drench but not foliar spray after flowering is initiated;
(ii) the AVG application comprises root drench prior to flowering and separately comprises foliar spray after flowering is initiated,
optionally, wherein the AVG application comprises at least one root drench application without foliar spray prior to flowering, further optionally, wherein the AVG application comprises root drench but not foliar spray prior to flowering, and/or
optionally, wherein the AVG application separately comprises at least one foliar spray application without root drench after flowering is initiated, further optionally, wherein the AVG application comprises foliar spray but not root drench after flowering is initiated;
(iii) the AVG application comprises foliar spray prior to flowering and separately comprises root drench after flowering is initiated,
optionally, wherein the AVG application comprises at least one foliar spray application without root drench prior to flowering, further optionally, wherein the AVG application comprises foliar spray but not root drench prior to flowering, and/or
optionally, wherein the AVG application separately comprises at least one root drench application without foliar spray after flowering is initiated, further optionally, wherein the AVG application comprises root drench and not foliar spray after flowering is initiated; or
(iv) the AVG application comprises foliar spray prior to flowering and separately comprises foliar spray after flowering is initiated,
optionally, wherein the AVG application comprises at least one foliar spray application without root drench prior to flowering, further optionally, wherein the AVG application comprises foliar spray but not root drench prior to flowering, and/or
optionally, wherein the AVG application separately comprises at least one foliar spray application without root drench after flowering is initiated, further optionally, wherein the AVG application comprises foliar spray and not root drench after flowering is initiated.

20. The method of any one of claims 1 to 19, wherein AVG is re-applied 2, 3, 4, 5, 6, 7, 8, or 9 days after the initial treatment,
optionally, wherein AVG is further re-applied 2, 3, 5, 6, 7, 8, or 9 days after the application that is 2, 3, 4, 5, 6, 7, 8, or 9 days after the initial treatment.

21. The method of any one of claims 1 to 20, wherein AVG is re-applied 10, 11, 12, 13, 14, 15, or 16 days after the initial treatment.

22. The method of any one of claims 1 to 22, wherein one the same day of or 1, 2, 3, 4, 5, 6, 7, 8, 9, 10, 11, 12, 13 or 14 days after the initial application of AVG to the plant, the plant is exposed to environmental conditions that induce flowering in Cannabis sativa,
optionally, wherein prior to the initial application of AVG to the plant, the plant is grown under vegetative growth conditions for Cannabis sativa,
optionally, wherein the plant is grown under vegetative growth conditions for Cannabis sativa up until 1, 2, 3, 4, 5, 6, or 7 days after the initial application of AVG to the plant, at which time the plant is switched to environmental conditions that induce flowering in Cannabis sativa.

23. The method of any one of claims 1 to 22, wherein AVG is applied in one or more applications by root drench in a solution containing 0.3 mg to 3.5 mg of AVG per millimeter of stem diameter wherein the AVG solution is applied at a volume of at least 25 mL, 50 mL, 100 mL, 150 mL, 200 mL, 400 mL:
optionally, wherein the volume of solution is sufficient to come in contact with a majority of the root area; and/or
optionally, wherein the volume of solution is sufficient to contact at least 50%, 60%, 70%, 80%, 90%, 95%, 98%, 99%, or 100% of the root area.

24. The method of any one of claims 1 to 23, wherein at least 5, 10, 25, 50, 100, 125, 150, 200, 250, 500, 750, 1,000, 1,250, 1,500, 2,000, or 3,000 genetically female Cannabis sativa seeds are produced on a self-pollinated plant.

25. The method of claim 24, wherein all the seeds produced on the self-pollinated plant are genetically female.

26. The method of any one of claims 1 to 23, wherein at least 5, 10, 25, 50, 100, 125, 150, 200, 250, 500, 750, 1,000, 1,250, 1,500, 2,000, or 3,000 genetically female Cannabis sativa seeds are produced on a cross-pollinated plant.

27. The method of claim 26, wherein all the seeds produced on the cross-pollinated plant are genetically female.

28. The method of any one of claims 1 to 27, wherein exclusively genetically female Cannabis sativa seed is produced on a commercial scale;
optionally, wherein at least 5,000, 10,000, 20,000, 50,000, 100,000, 500,000, or 1,000,000 exclusively genetically female Cannabis sativa seeds are produced.

29. The method of any one of claims 1 to 28, further comprising growing a Cannabis sativa plant from the genetically female Cannabis sativa seed produced.

30. The method of claim 29, comprising growing a population of Cannabis sativa plants.

31. The method of claim 30, wherein the population is exclusively genetically female.

32. The method of claim 30 or 31, wherein the population comprises at least about 6 plants, 12 plants, 24 plants, 48 plants, 72 plants, 96 plants, 200 plants, 500 plants, 1,000 plants, 5,000 plants, 10,000 plants, 50,000 plants, or 100,000 plants.

33. A floral bud/inflorescence of the Cannabis sativa plant of claim 29.

34. A seed of the Cannabis sativa plant of claim 29,
optionally wherein the seed is part of a seed lot.

35. A Cannabis sativa plant grown from the seed of claim 34 or a progeny thereof.

36. A population of Cannabis sativa plants grown from a plurality of seeds of claim 34,
optionally, wherein all of the plants in the population are genetically female plants.

37. The population of claim 36, wherein the population comprises at least about 6 plants, 12 plants, 24 plants, 48 plants, 72 plants, 96 plants, 200 plants, 500 plants, 1,000 plants, 5,000 plants, 10,000 plants, 50,000 plants, or 100,000 plants.

38. A method of inducing male flowers on a genetically female Cannabis sativa plant, the method comprising applying the chemical ethylene inhibitor aminoethoxyvinylglycine (AVG) to a genetically female Cannabis sativa plant,
wherein the female plant forms one or more pollen-producing fertile male flowers.

39. The method of claim 38, wherein (AVG) is applied to the plant in at least two, three, or four separate applications.

40. The method of claim 38 or 39, wherein (AVG) is applied in at least one application by root drench to the plant.

41. The method of any one of claims 38 to 40, wherein (AVG) is applied in at least one application by foliar spray to the plant.

42. The method of any one of claims 38 to 41, wherein (AVG) is applied to the plant and:
(i) wherein the application comprises root drench in all applications;
(ii) wherein the application comprises root drench but not foliar spray in any applications;
(iii) wherein the application comprises foliar spray in all applications;
(iv) wherein the application comprises foliar spray but not root drench in any applications;
(v) wherein the application comprises root drench combined with foliar spray in all applications;
(vi) wherein the application comprises root drench combined with foliar spray in at least one application and root drench but not foliar spray in at least one separate application;
(vii) wherein the application comprises root drench combined with foliar spray in at least one application and foliar spray but not root drench in at least one separate application; and/or
(viii) wherein the application comprises root drench but not foliar spray in at least one application and also comprises foliar spray but not root drench in at least one separate application.

43. The method of claim 42, wherein in the first application of AVG to the plant comprises application by root drench;
optionally, wherein the first application of AVG to the plant comprises root drench but not foliar spray.

44. The method of claim 42, wherein the first application of AVG to the plant comprises application by foliar spray;
optionally, wherein the first application of AVG to the plant comprises foliar spray but not root drench.

45. The method of any one of claims 38 to 44, wherein the AVG is applied to the plant prior to flowering;
optionally, wherein AVG is first applied to the plant prior to flowering;
optionally, wherein AVG is only applied to the plant prior to flowering.

46. The method of claim 45, wherein AVG is applied to the plant in at least two, three, or four separate applications prior to flowering.

47. The method of any one of claims 38 to 46, wherein AVG is applied to the plant after flowering is initiated,
optionally, wherein AVG is applied to the plant in at least two, three, or four separate applications after flowering is initiated,
optionally, wherein AVG is only applied to the plant after flowering is initiated.

48. The method of any one of claims 38 to 47, wherein AVG is applied to the plant both prior to flowering and after flowering is initiated.

49. The method of claim 48, wherein:
(i) the AVG application comprises root drench prior to flowering and separately comprises root drench after flowering is initiated,
optionally, wherein the AVG application comprises at least one root drench application without foliar spray prior to flowering, further optionally, wherein the AVG application comprises root drench but not foliar spray prior to flowering, and/or
optionally, wherein the AVG application separately comprises at least one root drench application without foliar spray after flowering is initiated, further optionally, wherein the AVG application comprises root drench but not foliar spray after flowering is initiated;
(ii) the AVG application comprises root drench prior to flowering and separately comprises foliar spray after flowering is initiated,
optionally, wherein the AVG application comprises at least one root drench application without foliar spray prior to flowering, further optionally, wherein the AVG application comprises root drench but not foliar spray prior to flowering, and/or
optionally, wherein the AVG application separately comprises at least one foliar spray application without root drench after flowering is initiated, further optionally, wherein the AVG application comprises foliar spray but not root drench after flowering is initiated;
(iii) the AVG application comprises foliar spray prior to flowering and separately comprises root drench after flowering is initiated,
optionally, wherein the AVG application comprises at least one foliar spray application without root drench prior to flowering, further optionally, wherein the AVG application comprises foliar spray but not root drench prior to flowering, and/or
optionally, wherein the AVG application separately comprises at least one root drench application without foliar spray after flowering is initiated, further optionally, wherein the AVG application comprises root drench and not foliar spray after flowering is initiated; or
(iv) the AVG application comprises foliar spray prior to flowering and separately comprises foliar spray after flowering is initiated,
optionally, wherein the AVG application comprises at least one foliar spray application without root drench prior to flowering, further optionally, wherein the AVG application comprises foliar spray but not root drench prior to flowering, and/or
optionally, wherein the AVG application separately comprises at least one foliar spray application without root drench after flowering is initiated, further optionally, wherein the AVG application comprises foliar spray and not root drench after flowering is initiated.

50. The method of any one of claims 38 to 49, wherein AVG is re-applied 2, 3, 4, 5, 6, 7, 8, or 9 days after the initial treatment,
optionally, wherein AVG is further re-applied 2, 3, 4, 5, 6, 7, 8, or 9 days after the application that is 2, 3, 4, 5, 6, 7, 8, or 9 days after the initial treatment.

51. The method of any one of claims 38 to 50, wherein AVG is re-applied 10, 11, 12, 13, 14, 15, or 16 days after the initial treatment.

52. The method of any one of claims 38 to 51, wherein on the same day of or 1, 2, 3, 4, 5, 6, 7, 8, 9, 10, 11, 12, 13, or 14 days after the initial application of AVG to the plant, the plant is exposed to environmental conditions that induce flowering in Cannabis sativa,
optionally, wherein prior to the initial application of AVG to the plant, the plant is grown under vegetative growth conditions for Cannabis sativa,
optionally, wherein the plant is grown under vegetative growth conditions for Cannabis sativa up until 1, 2, 3, 4, 5, 6, or 7 days after the initial application of AVG to the plant, at which time the plant is switched to environmental conditions that induce flowering in Cannabis sativa.

53. The method of any one of claims 38 to 52, wherein AVG is applied in one or more applications by root drench in a solution containing 0.3 mg to 3.5 mg of AVG per millimeter of stem diameter wherein the AVG solution is applied at a volume of at least 25 mL, 50 mL, 100 mL, 150 mL, 200 mL, 400 mL;
optionally, wherein the volume of solution is sufficient to come in contact with a majority of the root area;
optionally, wherein the volume of solution is sufficient to contact at least 50%, 60%, 70%, 80%, 90%, 95%, 98%, 99%, or 100% of the root area.

54. A method of producing an inbred variety of Cannabis sativa, the method comprising:
(i) producing a genetically female Cannabis sativa plant with induced-male flowers, wherein the female plant comprises one or more pollen-producing fertile male flowers, by the method of any one of claims 38 to 53;
(ii) producing seed by:
(a) self-pollination of pollen from an induced-male flower onto a female flower of the same plant, or
(b) cross-pollination of pollen from induced-male flowers onto female flowers of a separate but genetically identical female plant,
optionally, wherein the genetically identical female plant is produced through clonal propagation; and
(iii) growing a genetically female Cannabis sativa plant from a seed produced in (ii); optionally,
(iv) repeating steps (i), (ii), and (iii) for at least two, three, four, five, six, or seven generations.

55. An inbred Cannabis sativa seed produced by the method of claim 54,
optionally wherein the seed is part of a seed lot.

56. An inbred variety of Cannabis sativa, or a plant of the variety, produced by the method of claim 54.

57. A population of inbred Cannabis sativa plants comprising the inbred variety of claim 56.

58. The population of inbred Cannabis sativa plants of claim 57, wherein the population comprises at least about 6 plants, 12 plants, 24 plants, 48 plants, 72 plants, 96 plants, 200 plants, 500 plants, 1,000 plants, 5,000 plants, 10,000 plants, 50,000 plants, or 100,000 plants.

59. The population of inbred Cannabis sativa plants of claim 57 or claim 58, wherein all of the plants in the population are genetically female plants.

60. A floral bud/inflorescence of the Cannabis sativa plant of claim 56 or of a Cannabis sativa plant of the population of any one of claims 57 to 59.

61. An inbred seed from the Cannabis sativa variety or plant of claim 56,
optionally wherein the seed is part of a seed lot.

62. A method of producing reproducible hybrid Cannabis sativa seed, the method comprising crossing two distinct inbred Cannabis sativa varieties, wherein at least one inbred variety has been produced by the method of claim 54.

63. The method of claim 62, wherein both inbred varieties have been produced by the method of claim 54.

64. A hybrid Cannabis sativa seed produced by the method of claim 62 or claim 63,
optionally wherein the seed is part of a seed lot.

65. A hybrid Cannabis sativa plant grown from the seed of claim 64.

66. A population of hybrid Cannabis sativa plants grown from a plurality of seeds of claim 64.

67. The population of inbred Cannabis sativa plants of claim 66, wherein the population comprises at least about 6 plants, 12 plants, 24 plants, 48 plants, 72 plants, 96 plants, 200 plants, 500 plants, 1,000 plants, 5,000 plants, 10,000 plants, 50,000 plants, or 100,000 plants.

68. The population of inbred Cannabis sativa plants of claim 66 or claim 67, wherein all of the plants in the population are genetically female plants.

69. A floral bud/inflorescence of the Cannabis sativa plant of claim 65 or of a Cannabis sativa plant of the population of any one of claims 66 to 68.

70. A method of producing a population of Cannabis sativa plants, the method comprising:
i) producing a genetically female Cannabis sativa plant with induced-male flowers, wherein the female plant comprises one or more pollen-producing fertile male flowers, by the method of any one of claims 38 to 53;
(ii) producing seed by pollinating a female flower with pollen from the induced one or more male flowers; and
(iii) growing a population of Cannabis sativa plants from a plurality of seed produced in (ii).

71. The method of claim 70, wherein the seed produced in (ii) is produced by self-pollination of pollen from the induced one or more male flowers onto a female flower of the same plant.

72. The method of claim 70, wherein the seed produced in (ii) is produced by cross-pollination of pollen from the induced one or more male flowers onto a female flower of a separate female plant,
optionally, wherein the separate female plant used for cross-pollination is genetically identical to the plant with the induced male flowers or wherein the separate female plant used for cross-pollination has a genotype that is distinct from the plant with the induced male flowers,
optionally, wherein the separate female plant used having a genotype that is distinct from the plant with the induced male flowers is from a distinct inbred variety.

73. The method of any one of claims 70 to 72, wherein the population comprises at least about 6 plants, 12 plants, 24 plants, 48 plants, 72 plants, 96 plants, 200 plants, 500 plants, 1,000 plants, 5,000 plants, 10,000 plants, 50,000 plants, or 100,000 plants.

74. The method of any one of claim 70 or 73, wherein all of the plants in the population are genetically female plants.

75. A method of producing inbred Cannabis sativa seed, the method comprising producing a plurality of genetically female Cannabis sativa plants with induced-male flowers, wherein the female plants comprise one or more pollen-producing fertile male flowers, by the method of any one of claims 38 to 53,
wherein the female plants with induced-male flowers are grown in isolation or in coincidence with non-induced female plants of the same genotype.

76. The method of producing Cannabis sativa seed of claim 75, wherein said method is used to produce seed on a production scale.

77. The method of producing Cannabis sativa seed claim 75 or 76, wherein pollen from the induced-male flowers: (a) naturally dehiscesses; (b) is collected for immediate application to female flowers; or (c) is stored for later application.

78. The method of claim 75 or 76, wherein the pollen from the induced-male flowers is disbursed for pollination by an ultrasonic dehiscence system.

79. A method of producing hybrid Cannabis sativa seed, the method comprising producing a plurality of genetically female Cannabis sativa plants with induced-male flowers, wherein the female plants comprise one or more pollen-producing fertile male flowers, by the method of any one of claims 38 to 53,
wherein the female plants with induced-male flowers are grown in isolation or in coincidence with non-induced female plants having a different genotype,
optionally, wherein the non-induced female plant having a different genotype is an inbred plant;
optionally, wherein said seed is produced on a production scale.

80. The method of claim 79, wherein pollen from the induced-male flowers: (a) naturally dehiscesses; (b) is collected for immediate application to female flowers; or (c) is stored for later application.

81. The method of claim 79 or 80, wherein the pollen from the induced one or more male flowers is disbursed for pollination by an ultrasonic dehiscence system.

82. A method of screening Cannabis sativa germplasm to identify genotypes responsive to the induction of male flowers on genetically female plants, the method comprising applying the chemical ethylene inhibitor aminoethoxyvinylglycine (AVG) to a genetically female plant and determining the response of the plant with respect to the induction of male flowers;
optionally, determining whether the plant forms one or more pollen-producing fertile male flowers.

83. The method of claim 82, further comprising selecting a plant based on its response to the induction of male flowers.

84. The method of claim 83, further comprising genotyping the selected plant.

85. The method of claim 84, further comprising identifying an association between the genotype of the selected plant and its response to the induction of male flowers.

86. The method of claim 85, wherein the genotype is associated with responsiveness to the induction of male flowers.

87. The method of any one of claims 82 to 86, wherein the induction of male flowers on genetically female plants is done by the method of any one of claims 38 to 53.

88. The method of any one of claims 82 to 87, further comprising treating a genetically female Cannabis sativa plant to induce male flowers, wherein the treated plant comprises a genotype associated with responsiveness to the induction of male flowers.

89. The method of any one of claims 82 to 88, further comprising self-pollinating a Cannabis sativa plant comprising a genotype associated with responsiveness to the induction of male flowers or crossing a first Cannabis sativa plant comprising a genotype associated with responsiveness to the induction of male flowers to a second plant.

90. The method of claim 89, wherein the second plant does not comprise the same genotype associated with responsiveness to the induction of male flowers as the first plant.

91. The method of claim 88 or 89, further comprising generating a population of Cannabis sativa plants segregating for a genotype associated with responsiveness to the induction of male flowers.

92. A method of introgressing a genotype associated with responsiveness to the induction of male flowers into plant germplasm using a genotype identified by the method of any one of claims 82 to 87.

93. A method of establishing inbred breeding pools of Cannabis sativa germplasm based on the genotypes identified by the method of any one of claims 82 to 87.

94. The method of claim 93, wherein the identified genotypes are associated with responsiveness to the induction of male flowers on female plants.

95. The method of claim 93 or claim 94, wherein the inbred breeding pools are used in hybrid development.

96. The method of any one of claims 82 to 95 wherein a Cannabis sativa plant comprising a genotype associated with responsiveness to the induction of male flowers is selected for use and/or treatment based on knowledge of such genotype.

97. A method of processing pollen from a genetically female plant with induced-male flowers produced by the method of any one of claims 38 to 53, the method comprising:
(i) removing an inflorescence-containing male flower from the female plant after mature male flowers or anthers are formed;
(ii) drying the male flowers or anthers for 1 to 5 days; and then
(iii) sifting to isolate pollen,
optionally where the isolated pollen is for immediate use or for storage.

98. In any of the above claims, wherein at least one Cannabis sativa plant is of a variety selected from the group consisting of Wily Jack, Birthday Cake, Mimosa, Gorilla Glue, Capstone, Sour Strawberry, Strawanna, or BioDiesel.

99. A genetically female Cannabis sativa plant comprising induced fertile male flowers produced by the method of any one of claims 38 to 54.

100. A genetically female Cannabis sativa seed, wherein the seed is obtained by the method of any one of claims 1 to 27.

101. The genetically female Cannabis sativa seed of claim 100, wherein the seed produced is inbred or wherein the seed produced is a hybrid.

102. A population of Cannabis sativa plants grown from a plurality of seeds of claim 100 or 101,
optionally wherein the population comprises at least about 6 plants, 12 plants, 24 plants, 48 plants, 72 plants, 96 plants, 200 plants, 500 plants, 1,000 plants, 5,000 plants, 10,000 plants, 50,000 plants, or 100,000 plants,
optionally, wherein all of the plants in the population are genetically female plants.

103. A floral bud/inflorescence of the Cannabis sativa plant of claim 99 or of a Cannabis sativa plant of the population of claim 102.

104. A method of growing a Cannabis sativa plant, the method comprising growing a Cannabis sativa plant from a genetically female Cannabis sativa seed of any one of claims 1 to 27.

105. A floral bud/inflorescence of the Cannabis sativa plant grown in the method of claim 104.

106. A population of exclusively female Cannabis sativa plants grown from seed, wherein the population of Cannabis sativa plants comprises at least about 6 plants, 12 plants, 24 plants, 48 plants, 72 plants, 96 plants, 200 plants, 500 plants, 1,000 plants, 5,000 plants, 10,000 plants, 50,000 plants, 100,000 plants, 500,000 plants, or 1,000,000 plants;
optionally, wherein the seed is inbred seed or wherein the seed is hybrid seed.
